(19) United States
(12) Patent Application Publication
(10) Pub. No.: US 2005/0013817 A1
(43) Pub. Date: Jan. 20, 2005

(54) Human SMAPK3-related gene variants associated with cancers

(76) Inventor: Ken-Shwo Dai, Hsinchu (TW)

Correspondence Address: Ladas & Parry, 26 West 61st Street, New York, NY, 10023, US

(21) Appl. No.: 10/623,108

(22) Filed: Jul. 18, 2003

(51) Int. Cl. A61K 39/395; C12Q 1/68; C07H 21/04; C12N 9/12

(52) U.S. Cl. 424/155.1; 435/6; 435/69.1; 435/194; 435/320.1; 435/325; 530/350; 536/23.2; 530/388.26

(57) ABSTRACT

The invention relates to the nucleic acid sequences of four novel human SMAPK3-related gene variants (SMAPK3V1, SMAPK3V2, SMAPK3V3 and SMAPK3V4) and the polypeptides encoded by SMAPK3V1 and SMAPK3V2. The invention also relates to the process for producing the polypeptides encoded by SMAPK3V1 and SMAPK3V2. The invention further relates to the use of the nucleic acid of SMAPK3V1, SMAPK3V2, SMAPK3V3 and SMAPK3V4 and the polypeptide encoded by SMAPK3V1 and SMAPK3V2 in diagnosing diseases associated with the deficiency of human SMAPK3V gene, in particular large cell lung cancers and Burkitt lymphoma.

### FIELD OF THE INVENTION

The invention relates to the nucleic acid sequences of four novel human SMAPK3-related gene variants (SMAPK3V1, SMAPK3V2, SMAPK3V3 and SMAPK3V4) and the polypeptides encoded thereby, the preparation process thereof, and the uses of the same in diagnosing diseases associated with the deficiency of the gene variants, in particular human cancers, e.g., large cell lung cancers and Burkitt lymphoma.

### BACKGROUND OF THE INVENTION

Lung cancer is one of the major causers of cancer-related deaths in the world. There are two primary types of lung cancers: small cell lung cancer (SCLC) and non-small cell lung cancer (NSCLC) (Carney, (1992a) Curr. Opin. Oncol. 4:292-8). Small cell lung cancer accounts for approximately 25% of lung cancer and spreads aggressively (Smyth et al. (1986) Q J Med. 61: 969-76; Carney, (1992b) Lancet 339: 843-6). Non-small cell lung cancer represents the majority (about 75%) of lung cancer and is further divided into three main subtypes: squamous cell carcinoma, adenocarcinoma, and large cell carcinoma (Ihde and Minutesna, (1991) Cancer 15: 105-54). In recent years, much progress has been made toward understanding the molecular and cellular biology of lung cancers. Many important contributions have been made by the identification of several key genetic factors associated with lung cancers. However, the treatments of lung cancers still mainly depend on surgery, chemotherapy, and radiotherapy. This is because the molecular mechanisms underlying the pathogenesis of lung cancers remain largely unclear.

A recent hypothesis suggests that lung cancer is caused by genetic mutations of at least 10 to 20 genes (Sethi, (1997) BMJ. 314: 652-655). Therefore, future strategies for the prevention and treatment of lung cancers will be focused on the elucidation of these genetic substrates, in particular, the genes associated with the regulation of cell proliferation. Many pathways are involved in the cell cycle regulation. Of these, the mitogen-activated protein kinase (MAPK), also termed extracellular signal-regulated kinase (ERK), signaling pathway is a pathway serves as an interface linking regulatory information generated at the cell surface to the gene expression that associated with cell cycle regulation (Blenis J, (1993) Proc Natl Acad Sci U S A 90:5889-92). The involvement of MAPK pathway in cell cycle regulation has been reported by several studies (Bauer et al. (2001) Proc Natl Acad Sci U S A 98: 12802-12807; Delmas et al. (2001) J Biol Chem 276:34958-65; Le Gall et al. (2000) Mol Biol Cell 11:1103-12). Recently, a novel protein (GenBank accession #AAH13992) isolated from Burkitt lymphoma showing its sequence similar to a component (MAPK3, also referred to as p44 MAPK) of MAPK signaling pathway, raised a possibility that the gene variants of this novel gene (we named it SMAPK3 for the purpose of the present study) may be important targets for diagnostic markers of cancers.

### SUMMARY OF THE INVENTION

The invention provides four SMAPK3-related gene variants found in human large cell lung carcinoma and pooled cancer tissues, respectively, and the polypeptide sequences encoded thereby, which are useful in the diagnosis of the diseases associated with the deficiency of human SMAPK3 gene, in particular cancers, preferably large cell lung carcinoma and Burkitt lymphoma.

The invention further provides an expression vector and host cell for expressing the polypeptides of SEQ ID NOs. 2 and 4.

The invention further provides a method for producing the polypeptides of SEQ ID NOs. 2 and 4.

The invention further provides an antibody specifically binding to the polypeptides of SEQ ID NOs. 2 and 4.

The invention also provides methods for diagnosing the diseases associated with the deficiency of human SMAPK3 gene, in particular cancers, preferable large cell lung carcinoma and Burkitt lymphoma.

### BRIEF DESCRIPTION OF THE DRAWINGS

FIGS. 1A to 1D show the nucleic acid sequence of SMAPK3V1 (SEQ ID NO: 1) and the amino acid sequence encoded thereby (SEQ ID NO:2).

FIGS. 2A to 2D show the nucleic acid sequence of SMAPK3V2 (SEQ ID NO:3) and the amino acid sequence encoded thereby (SEQ ID NO:4).

FIGS. 3A to 3E show the nucleic acid sequence of SMAPK3V3 (SEQ ID NO:5) and the amino acid sequence encoded thereby (SEQ ID NO:6).

FIGS. 4A to 4E show the nucleic acid sequence of SMAPK3V4 (SEQ ID NO:7) and the amino acid sequence encoded thereby (SEQ ID NO:8).

FIGS. 5A to 5D show the nucleotide sequence alignment between human SMAPK3 gene and SMAPK3V1, SMAPK3V2, SMAPK3V3 and SMAPK3V4.

FIGS. 6A to 6D show the amino acid sequence alignment among human SMAPK3 and the polypeptides encoded by SMAPK3V1, SMAPK3V2, SMAPK3V3 and SMAPK3V4.

### DETAILED DESCRIPTION OF THE INVENTION

According to the invention, all technical and scientific terms used have the same meanings as commonly understood by persons skilled in the art.

The term “antibody,” as used herein, denotes intact molecules (a polypeptide or group of polypeptides) as well as fragments thereof, such as Fab, R(ab′)₂, and Fv fragments, which are capable of binding the epitopic determinutesant. Antibodies are produced by specialized B cells after stimulation by an antigen. Structurally, antibody consists of four subunits including two heavy chains and two light chains. The internal surface shape and charge distribution of the antibody binding domain are complementary to the features of an antigen. Thus, antibody can specifically act against the antigen in an immune response.

The term “base pair (bp),” as used herein, denotes nucleotides composed of a purine on one strand of DNA which can be hydrogen bonded to a pyrimidine on the other strand. Thymine (or uracil) and adenine residues are linked by two hydrogen bonds. Cytosine and guanine residues are linked by three hydrogen bonds.

The term “Basic Local Alignment Search Tool (BLAST; Altschul et al., (1997) Nucleic Acids Res. 25: 3389-3402),” as used herein, denotes programs for evaluation of homologies between a query sequence (amino or nucleic acid) and a test sequence as described by Altschul et al. (Nucleic Acids Res. 25: 3389-3402, 1997). Specific BLAST programs are described as follows:
(1) BLASTN compares a nucleotide query sequence against a nucleotide sequence database;
(2) BLASTP compares an amino acid query sequence against a protein sequence database;
(3) BLASTX compares the six-frame conceptual translation products of a query nucleotide sequence against a protein sequence database;
(4) TBLASTN compares a query protein sequence against a nucleotide sequence database translated in all six reading frames; and
(5) TBLASTX compares the six-frame translations of a nucleotide query sequence against the six-frame translations of a nucleotide sequence database.

The term “cDNA,” as used herein, denotes nucleic acids that synthesized from a mRNA template using reverse transcriptase.

The term “cDNA library,” as used herein, denotes a library composed of complementary DNAs which are reverse-transcribed from mRNAs.

The term “complement,” as used herein, denotes a polynucleotide sequence capable of forming base pairing with another polynucleotide sequence. For example, the sequence 5′-ATGGACTTACT-3′ binds to the complementary sequence 5′-AGTAAGTCCAT-3′.

The term “deletion,” as used herein, denotes a removal of a portion of one or more amino acid residues/nucleotides from a gene.

The term “expressed sequence tags (ESTs),” as used herein, denotes short (200 to 500 base pairs) nucleotide sequence that derives from either 5′ or 3′ end of a cDNA.

The term “expression vector,” as used herein, denotes nucleic acid constructs which contain a cloning site for introducing the DNA into vector, one or more selectable markers for selecting vectors containing the DNA, an origin of replication for replicating the vector whenever the host cell divides, a terminator sequence, a polyadenylation signal, and a suitable control sequence which can effectively express the DNA in a suitable host. The suitable control sequence may include promoter, enhancer and other regulatory sequences necessary for directing polymerases to transcribe the DNA.

The term “host cell,” as used herein, denotes a cell which is used to receive, maintain, and allow the reproduction of an expression vector comprising DNA. Host cells are transformed or transfected with suitable vectors constructed using recombinant DNA methods. The recombinant DNA introduced with the vector is replicated whenever the cell divides.

The term “insertion” or “addition,” as used herein, denotes the addition of a portion of one or more amino acid residues/nucleotides to a gene.

The term “in silico,” as used herein, denotes a process of using computational methods (e.g., BLAST) to analyze DNA sequences.

The term “polymerase chain reaction (PCR),” as used herein, denotes a method which increases the copy number of a nucleic acid sequence using a DNA polymerase and a set of primers (about 20 bp oligonucleotides complementary to each strand of DNA) under suitable conditions (successive rounds of primer annealing, strand elongation, and dissociation).

The term “primer,” as used herein, denotes a single-stranded synthetic oligonucleotide designed to hybridize to a particular template DNA sequence. The forward primer is the one complementary to one strand at the 5′-end of the DNA sequence. The reverse primer is the one complementary to the other strand at the 3′-end of the DNA sequence.

The term “protein” or “polypeptide,” as used herein, denotes a sequence of amino acids in a specific order that can be encoded by a gene or by a recombinant DNA. It can also be chemically synthesized.

The term “nucleic acid sequence” or “polynucleotide,” as used herein, denotes a sequence of nucleotide (guanine, cytosine, thymine or adenine) in a specific order that can be a natural or synthesized fragment of DNA or RNA. It may be single-stranded or double-stranded.

The term “reverse transcriptase-polymerase chain reaction (RT-PCR),” as used herein, denotes a process which transcribes mRNA to complementary DNA strand using reverse transcriptase followed by polymerase chain reaction to amplify the specific fragment of DNA sequences.

The term “transformation,” as used herein, denotes a process describing the uptake, incorporation, and expression of exogenous DNA by prokaryotic host cells.

The term “transfection,” as used herein, a process describing the uptake, incorporation, and expression of exogenous DNA by eukaryotic host cells.

The term “variant,” as used herein, denotes a fragment of sequence (nucleotide or amino acid) inserted or deleted by one or more nucleotides/amino acids.

In the first aspect, the subject invention provides the nucleotide sequences of SMAPK3V1, SMAPK3V2, SMAPK3V3 and SMAPK3V4 and the polypeptides encoded by the two novel human SMAPK3-related gene variants and fragments thereof.

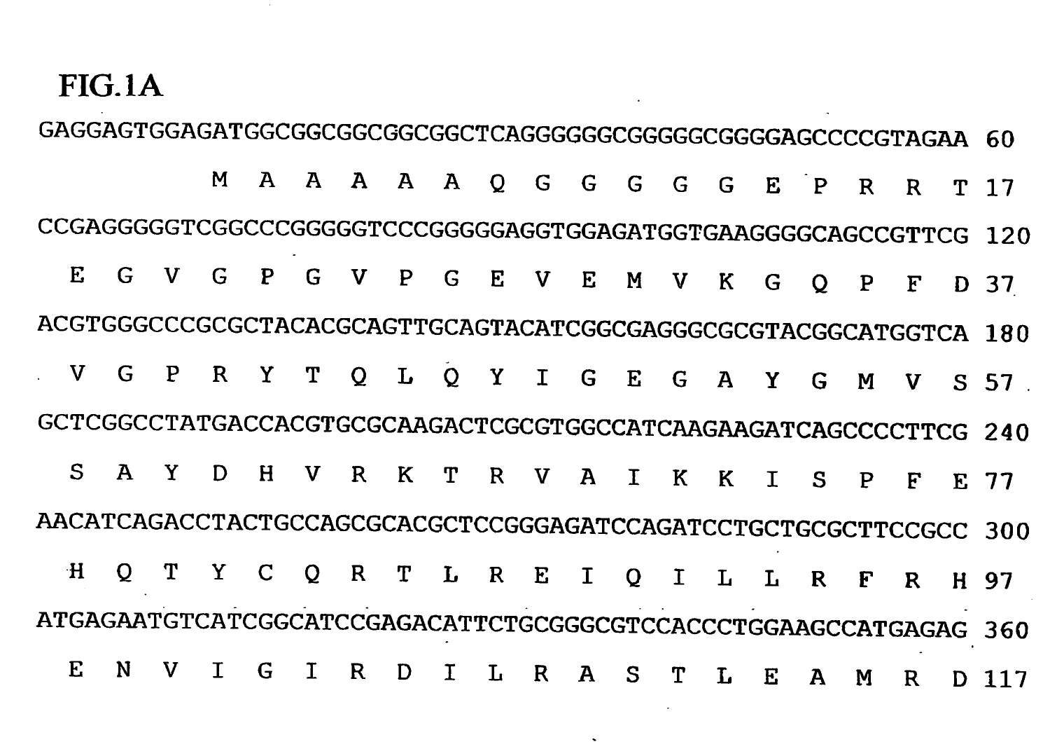

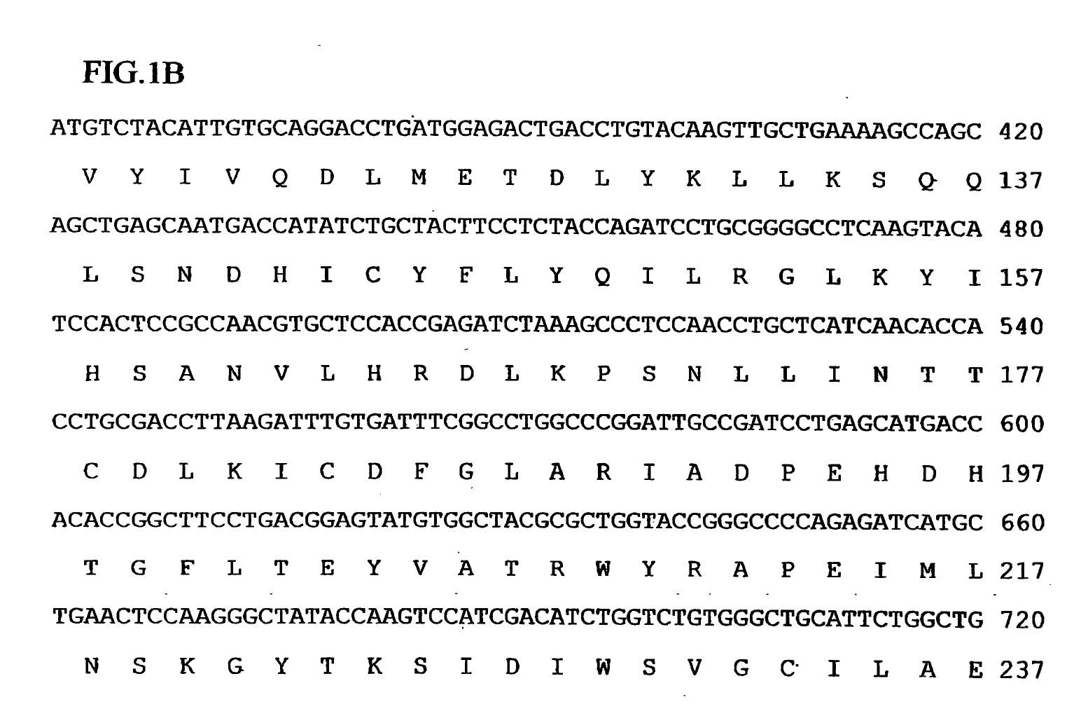

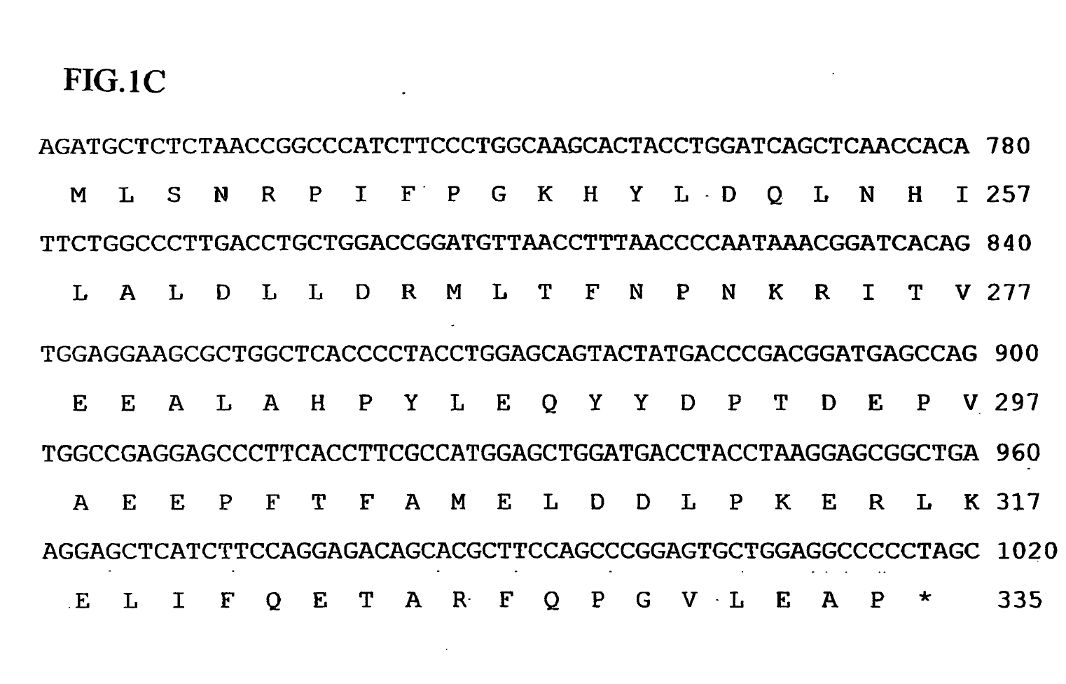

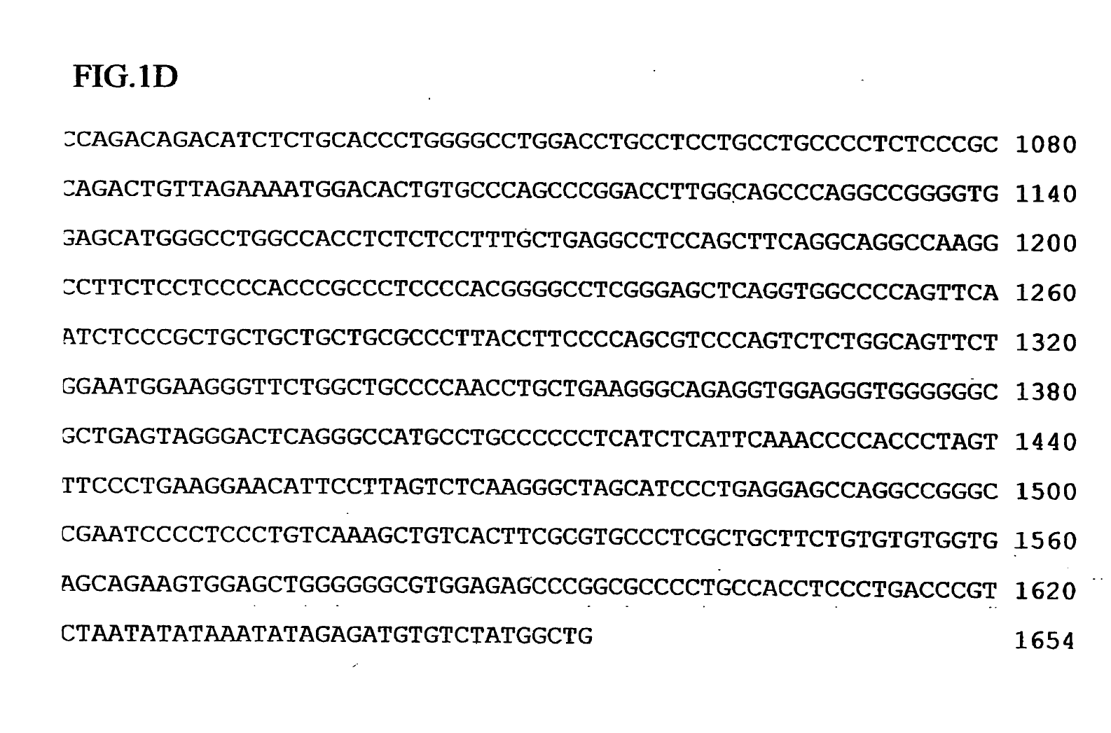

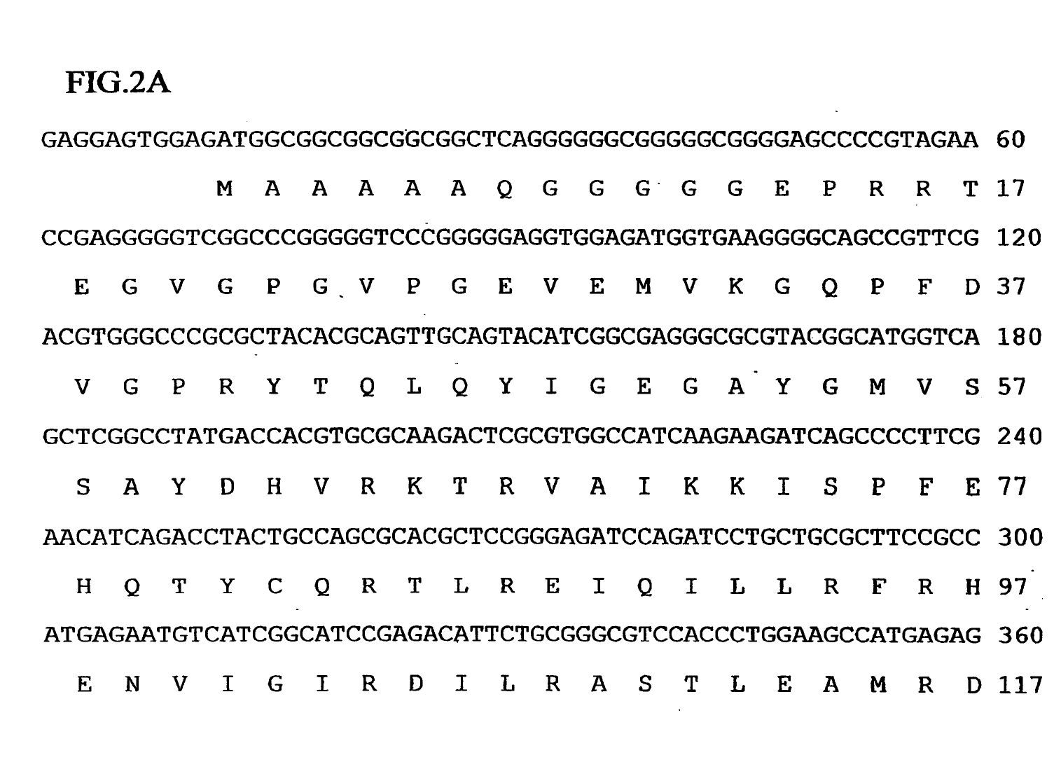

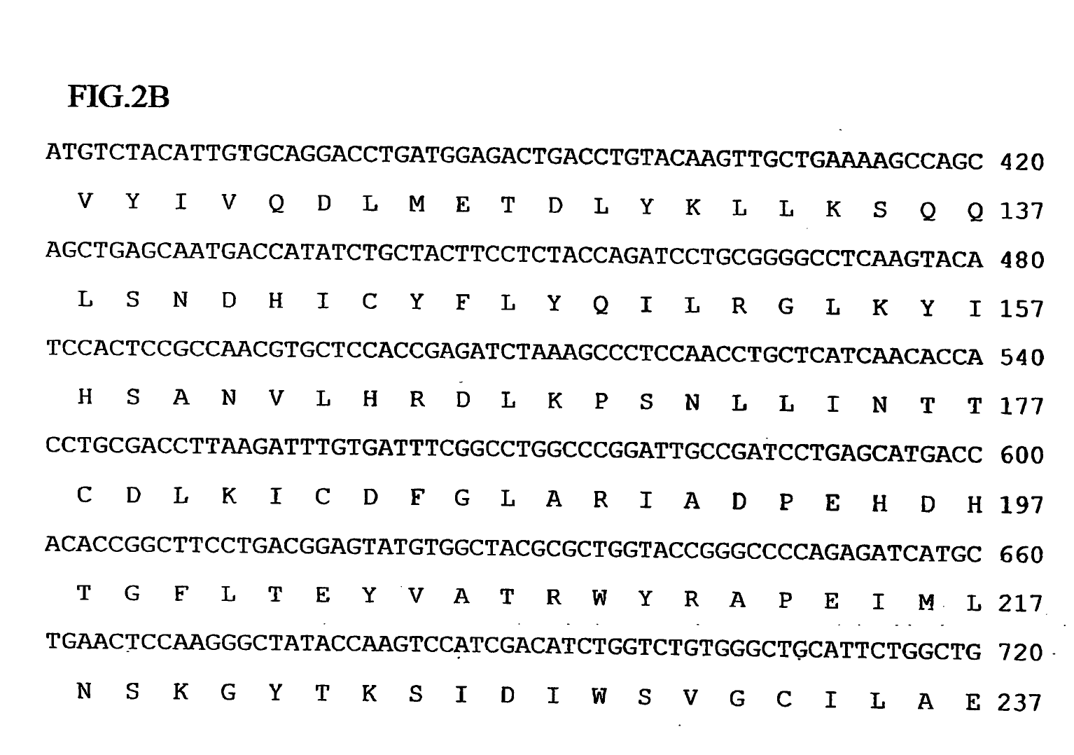

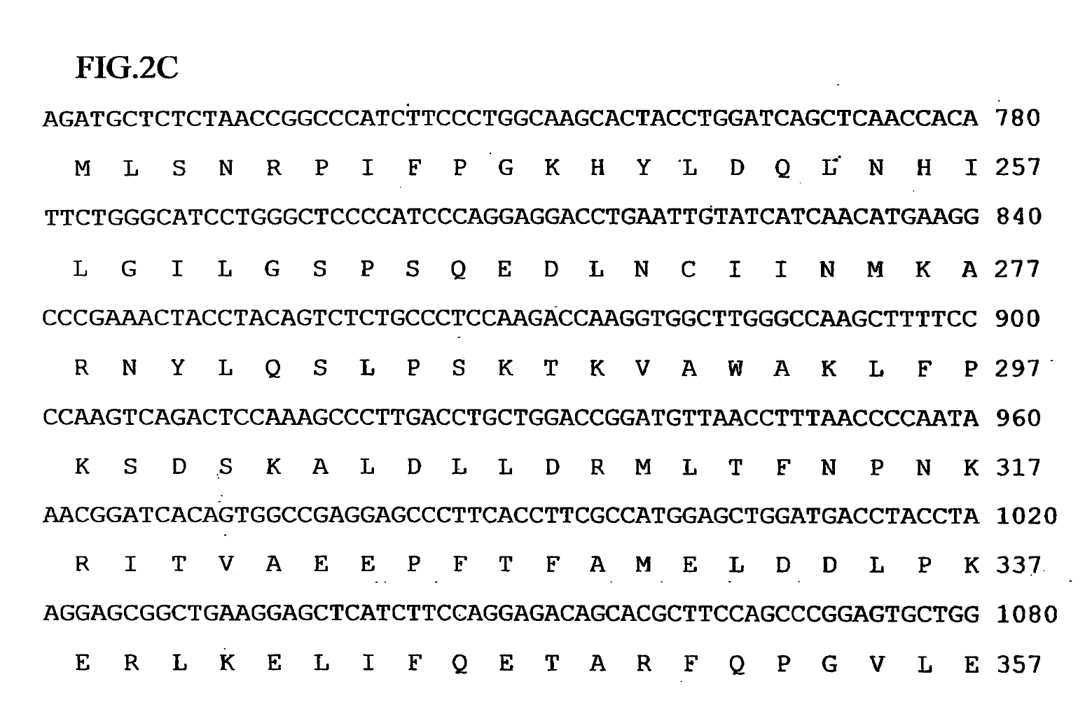

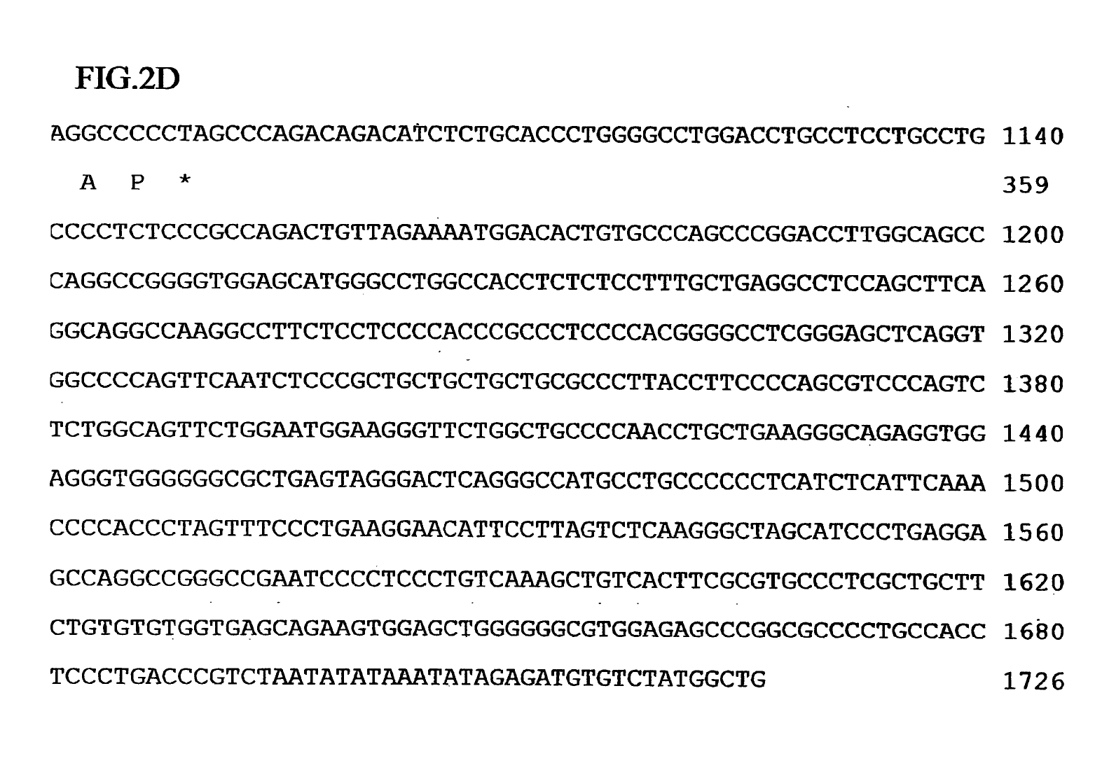

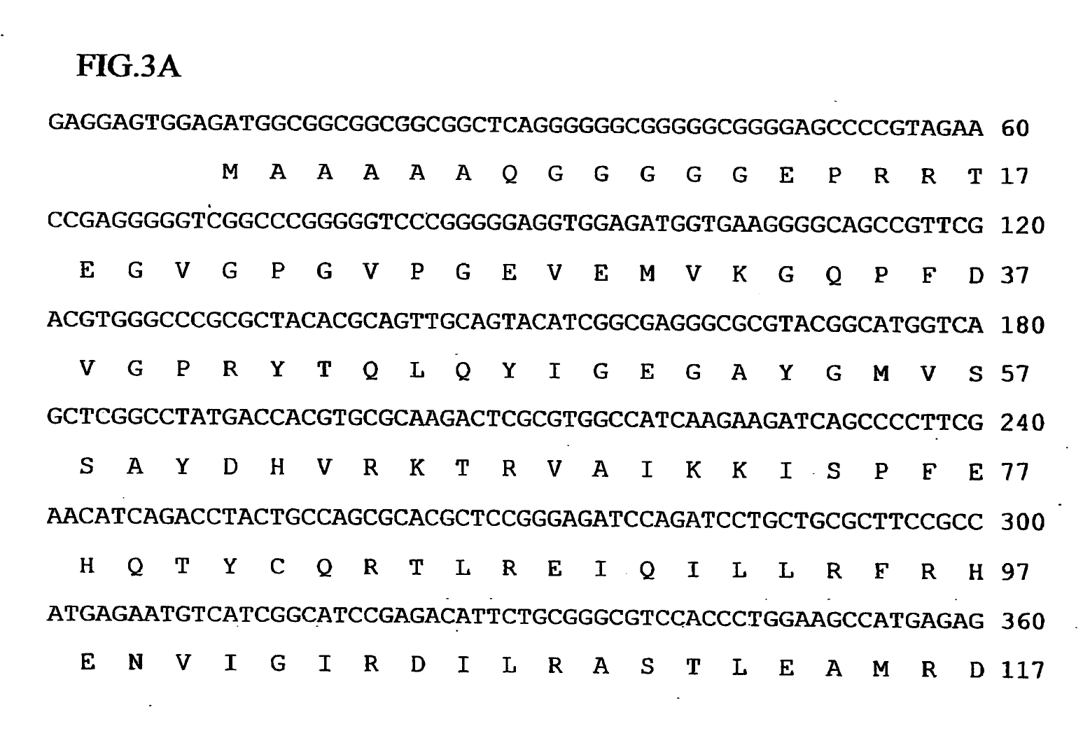

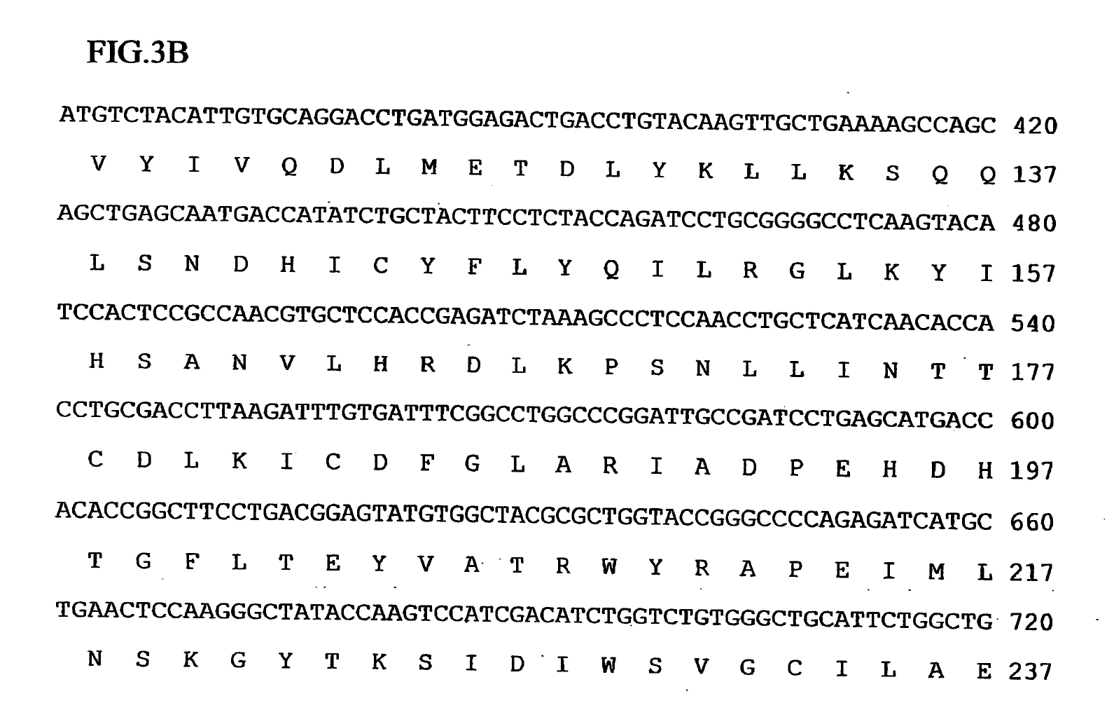

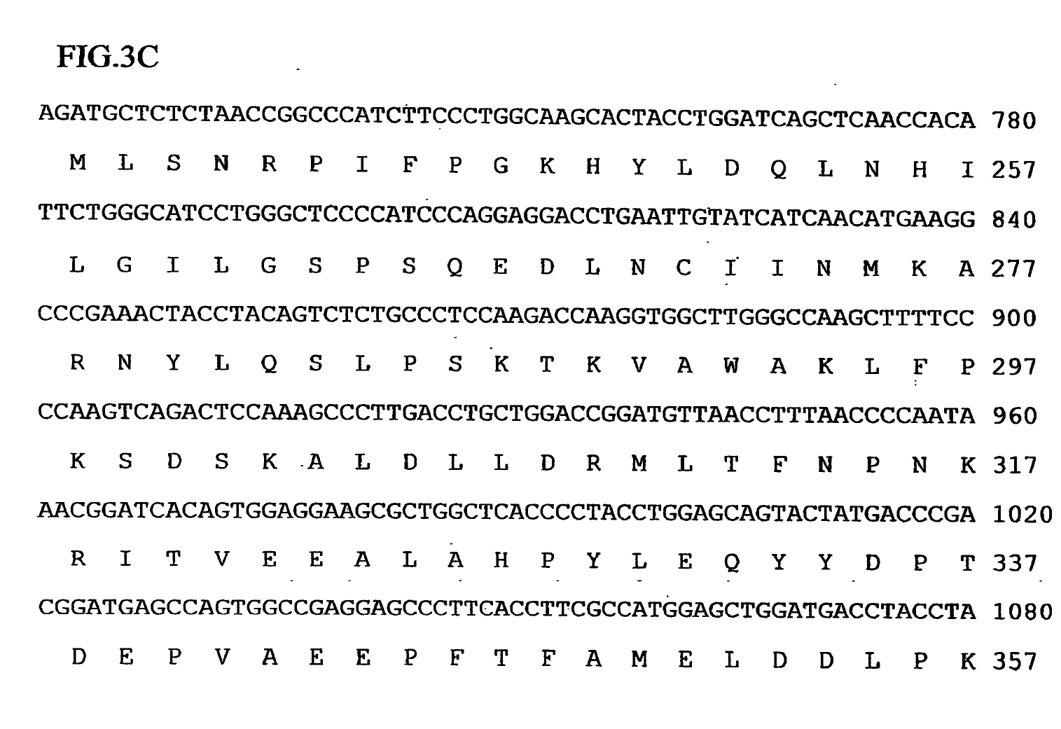

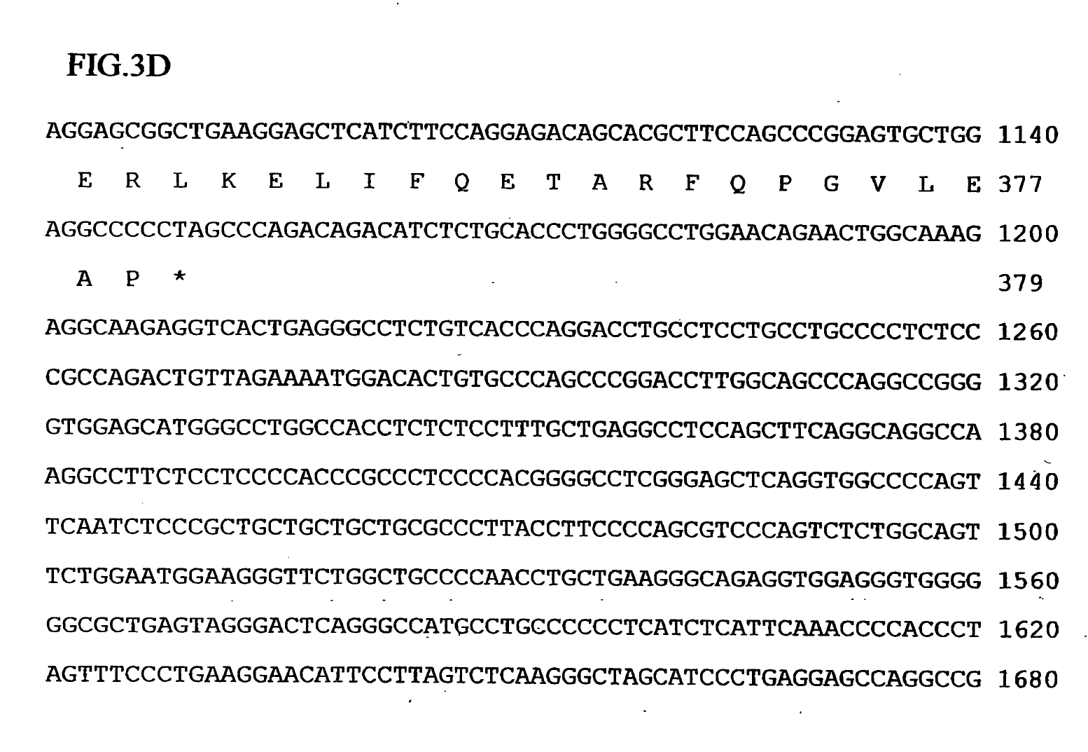

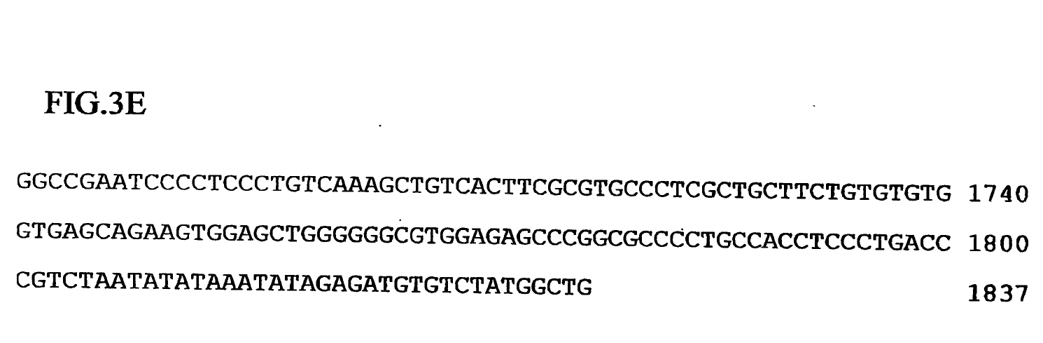

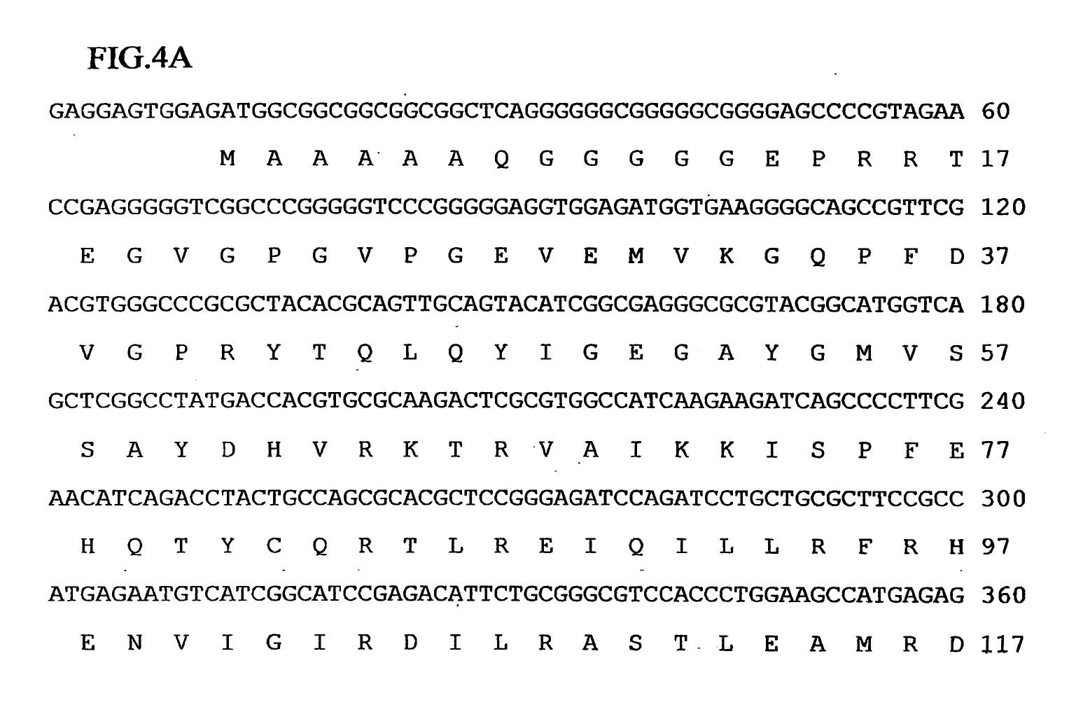

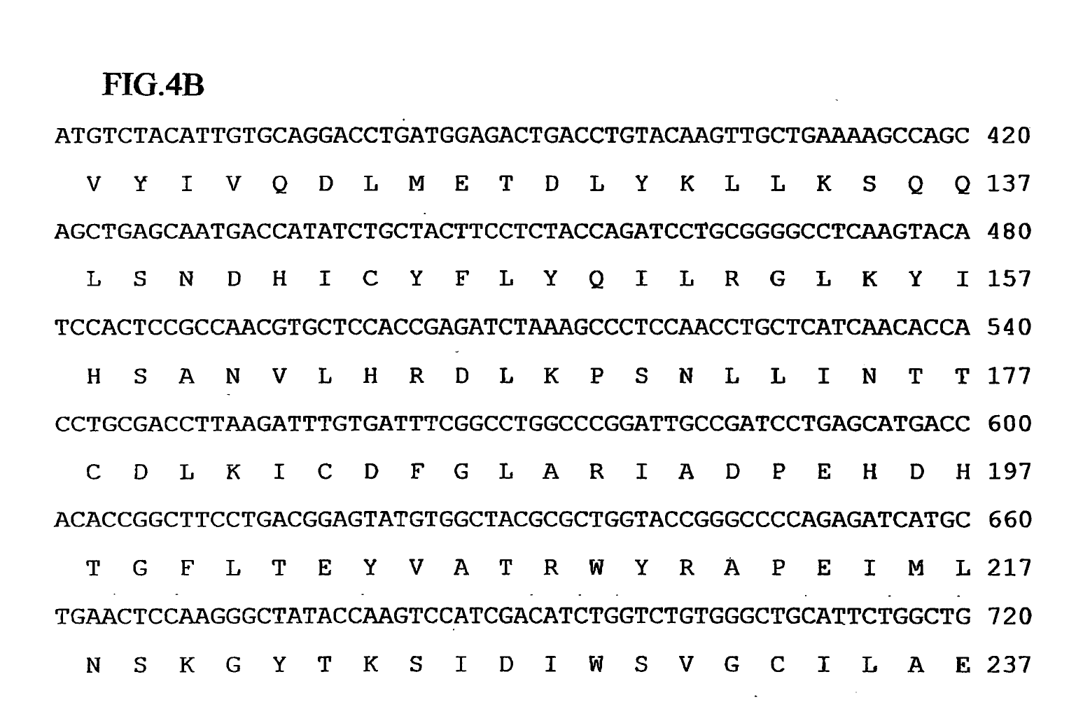

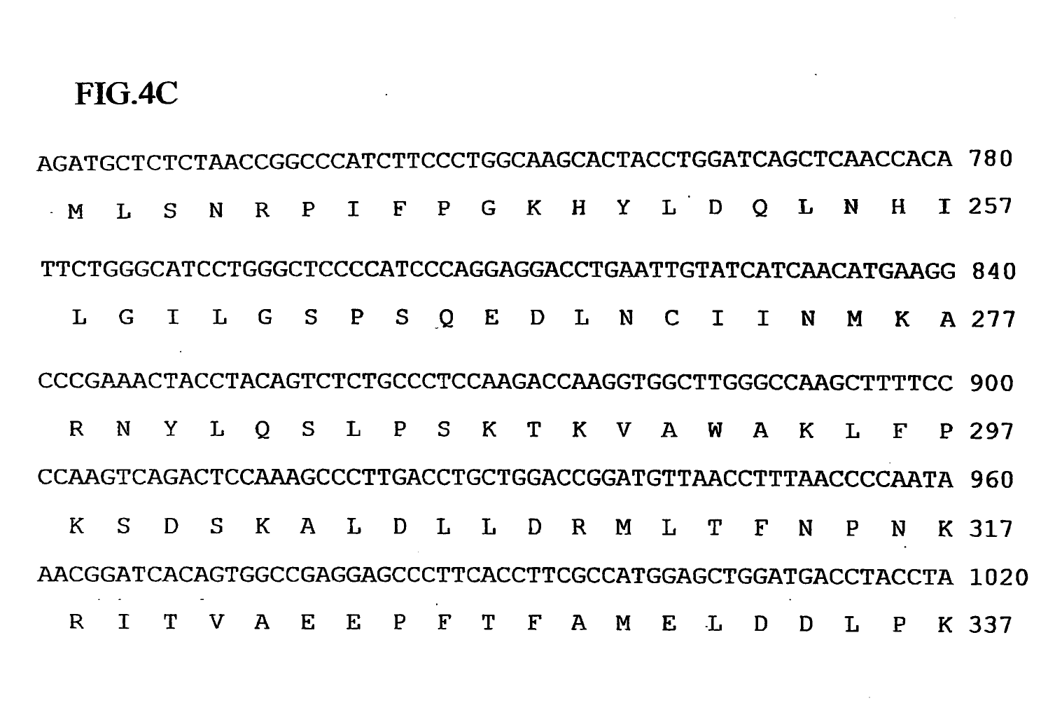

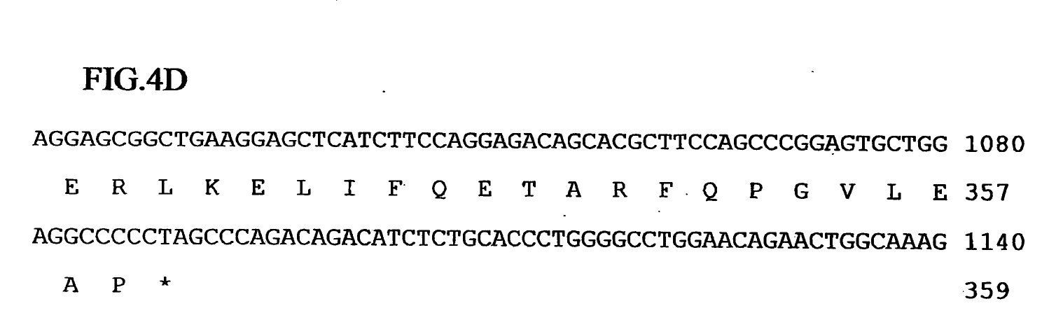

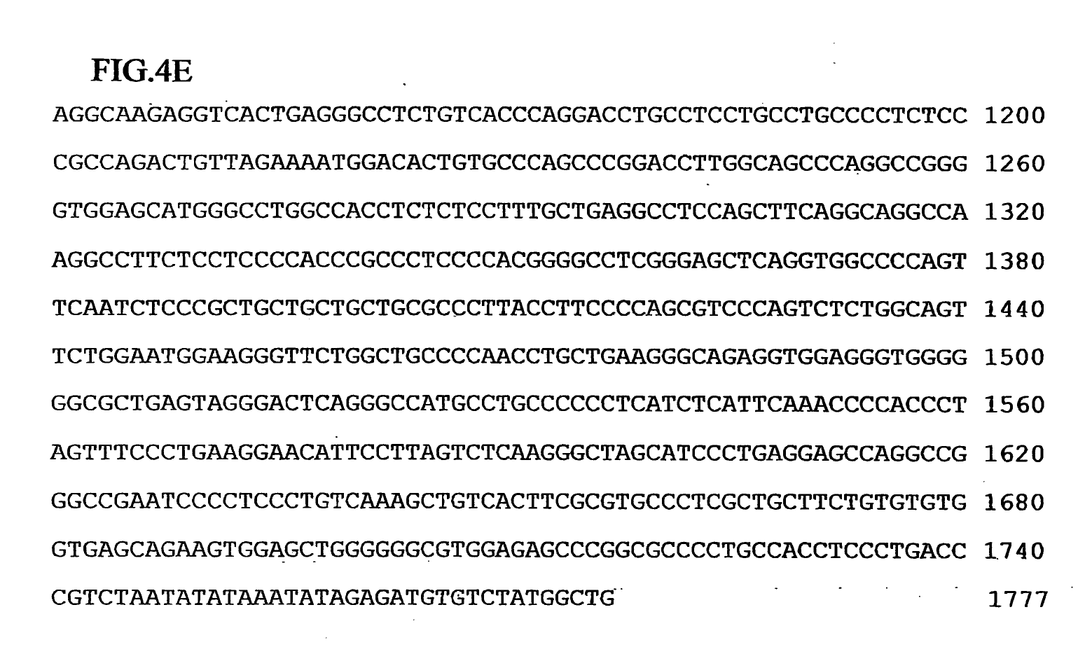

According to the invention, human SMAPK3 cDNA sequence was used to query our human EST databases (a normal lung, a large cell lung cancer, a squamous cell lung cancer, a small cell lung cancer, a Burkitt lymphoma, and a pooled cancer tissues) using BLAST program to search for SMAPK3-related gene variants. Four human cDNA partial sequences (i.e., ESTs) deposited in the databases showing similar to SMAPK3 were isolated and sequenced. These clones (named SMAPK3V1, SMAPK3V2, SMAPK3V3 and SMAPK3V4) were isolated from large cell lung cancer, Burkitt lymphoma, and pooled cancer tissues cDNA libraries, respectively. FIGS. 1, 2, 3 and 4 show the nucleic acid sequences (SEQ ID NOs: 1, 3, 5 and 7) of the variants (SMAPK3V1, SMAPK3V2, SMAPK3V3 and SMAPK3V4) and the corresponding amino acid sequences (SEQ ID NOs: 2, 4, 6 and 8) encoded thereby.

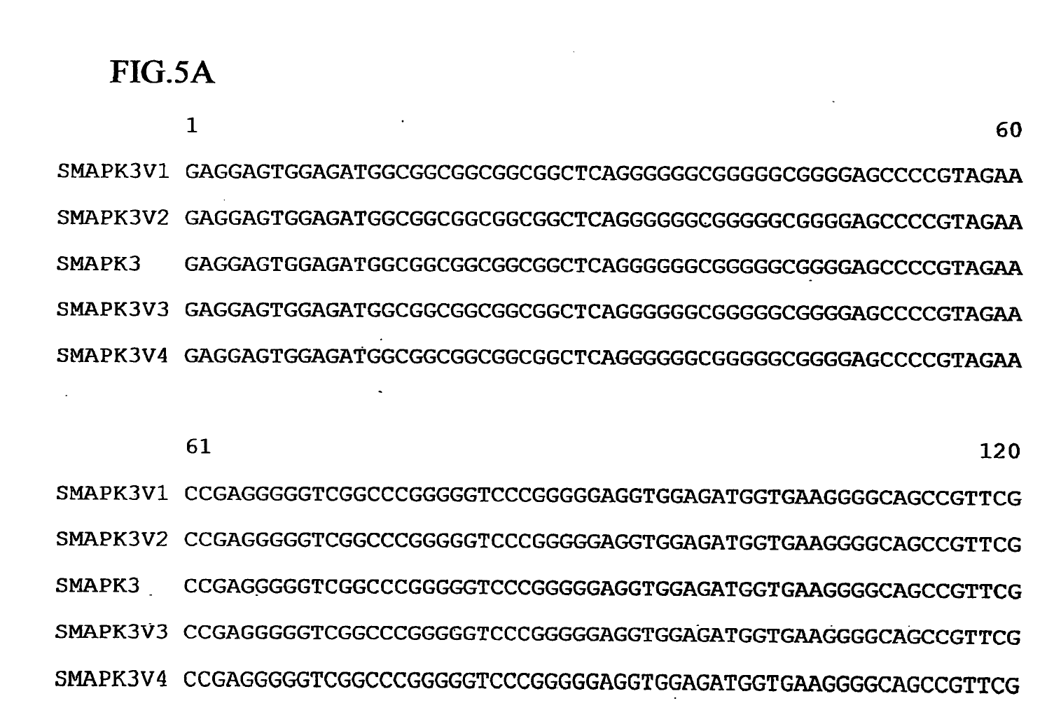

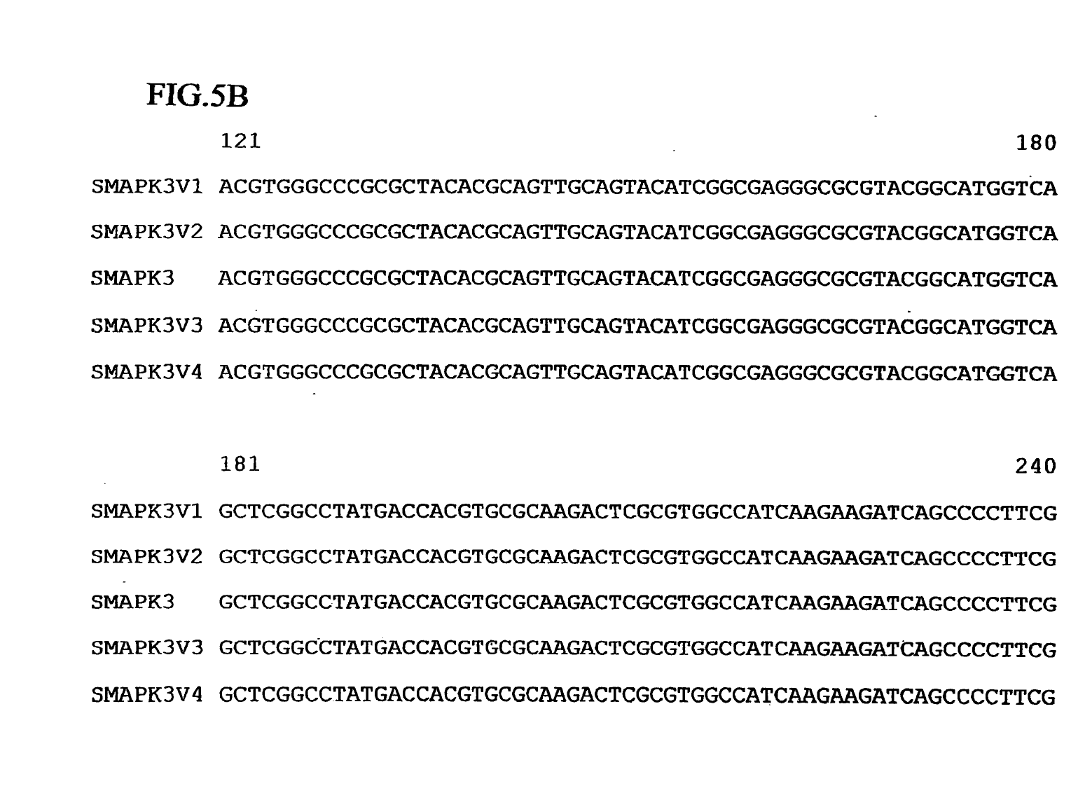

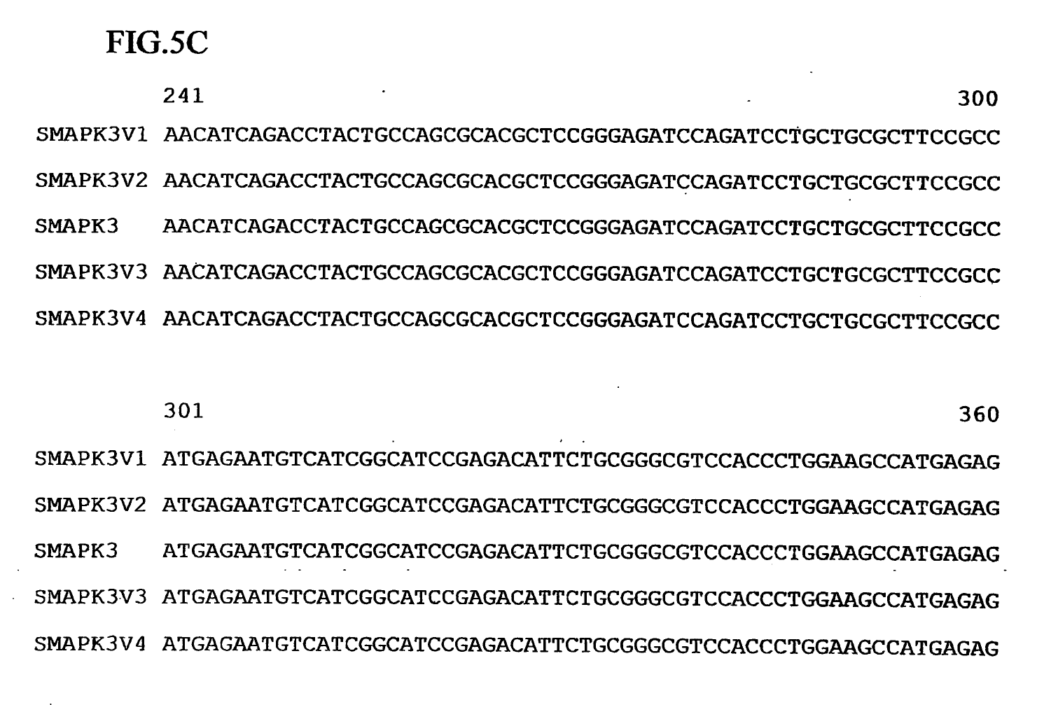

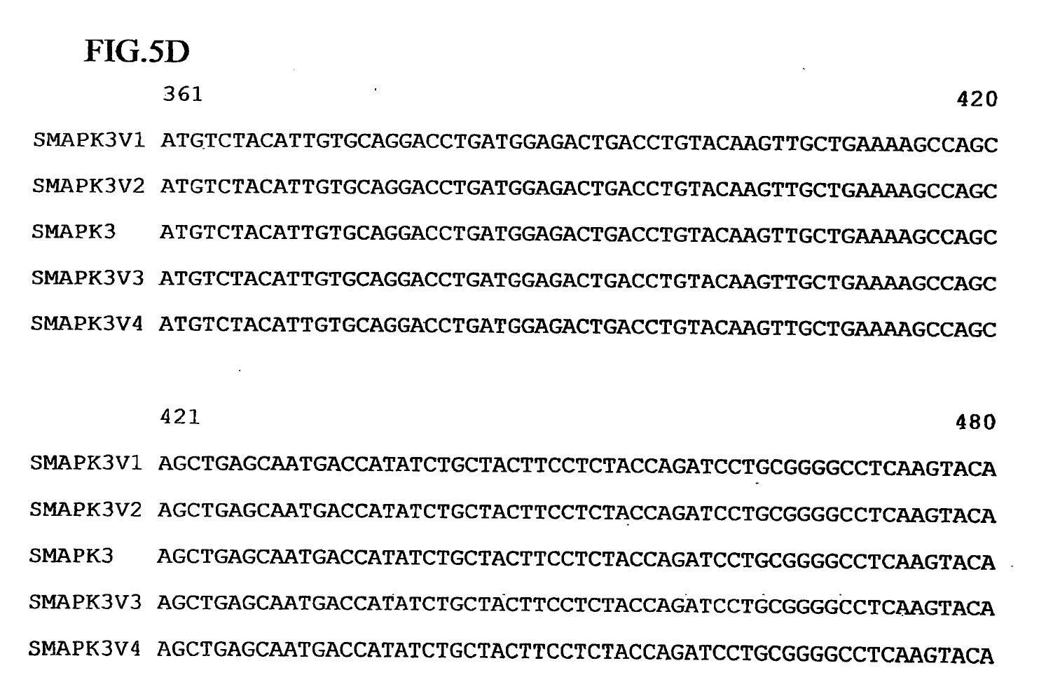

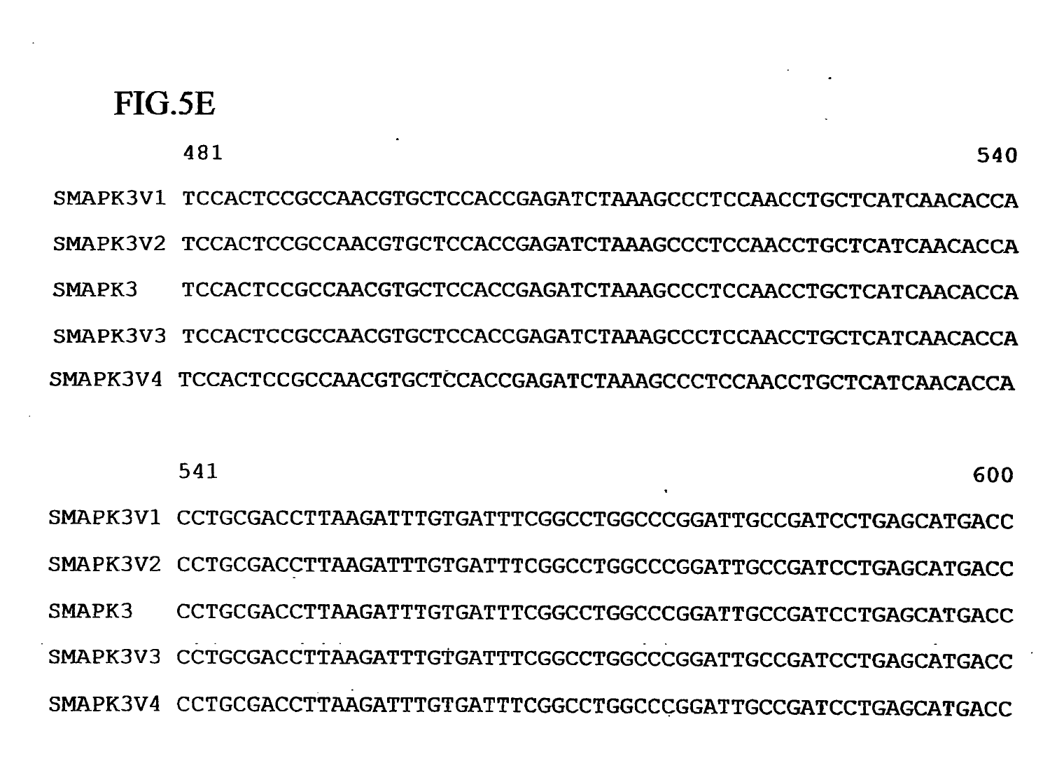

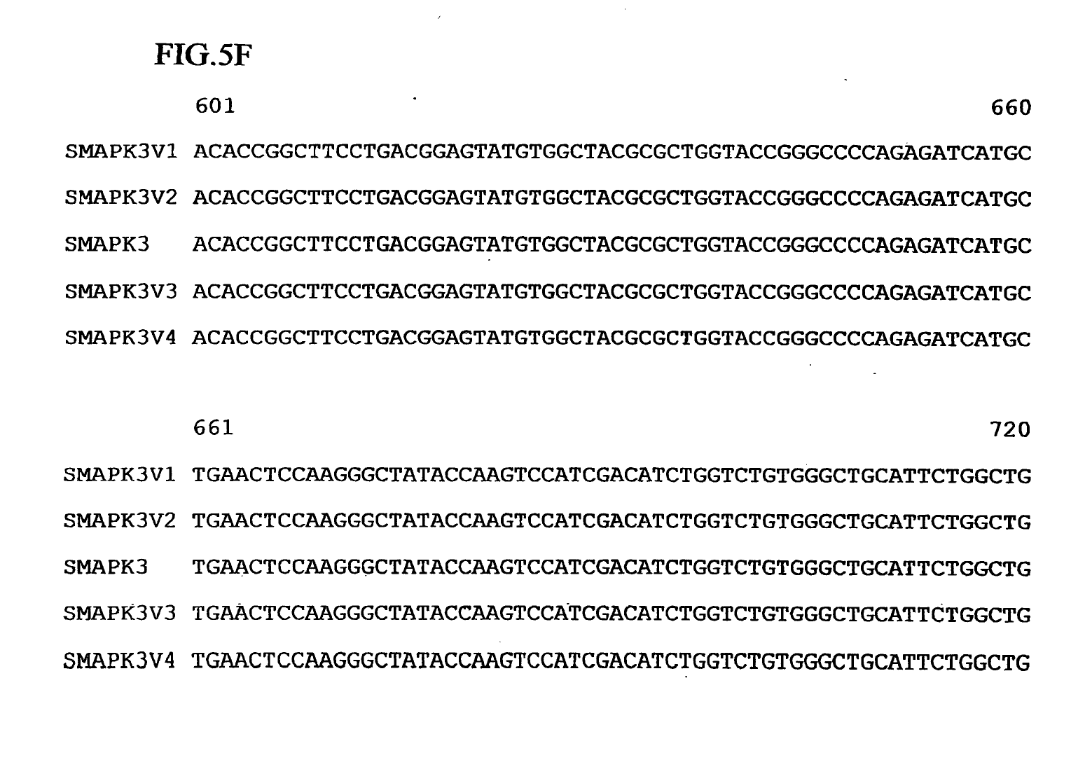

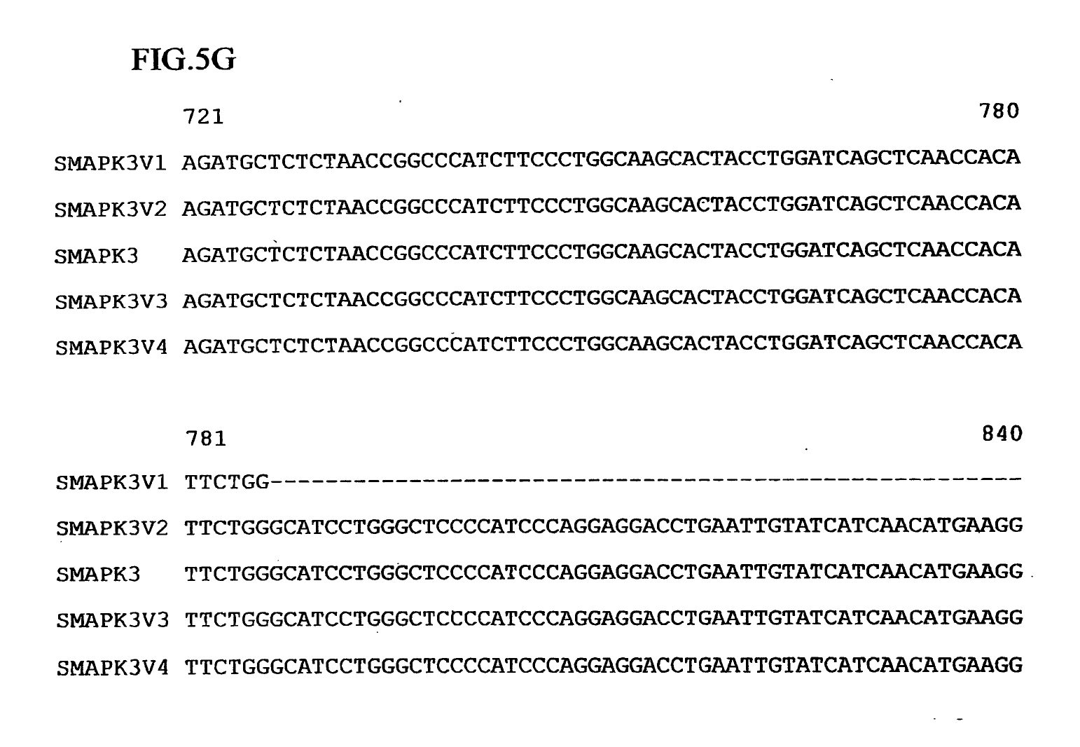

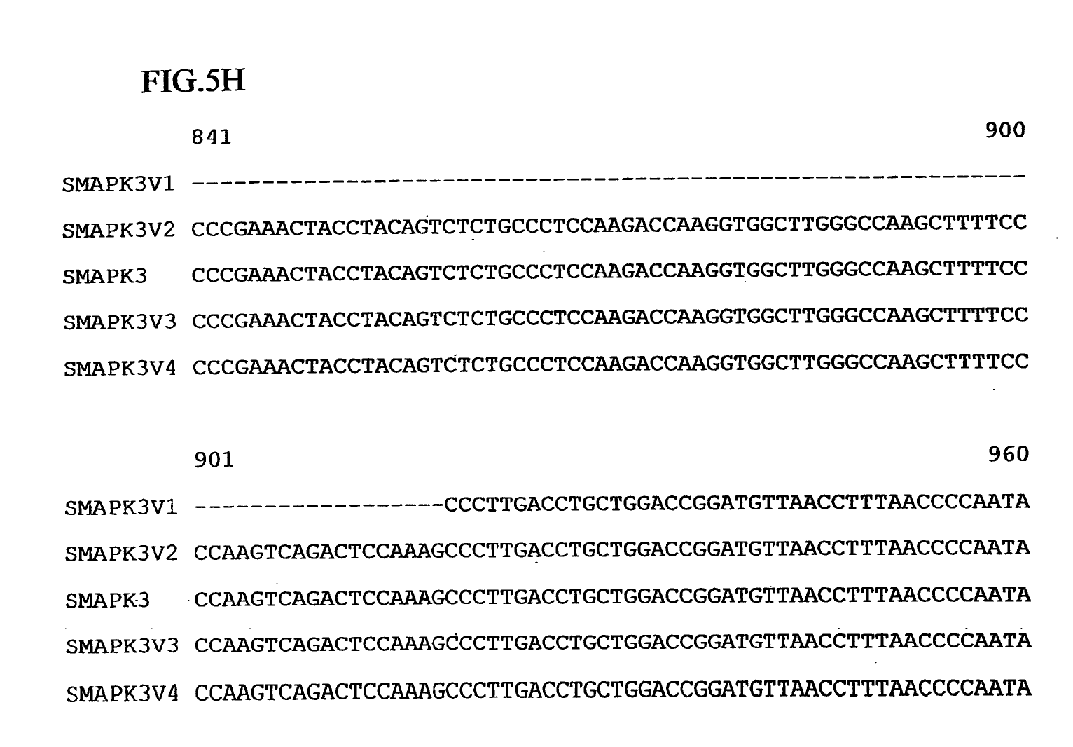

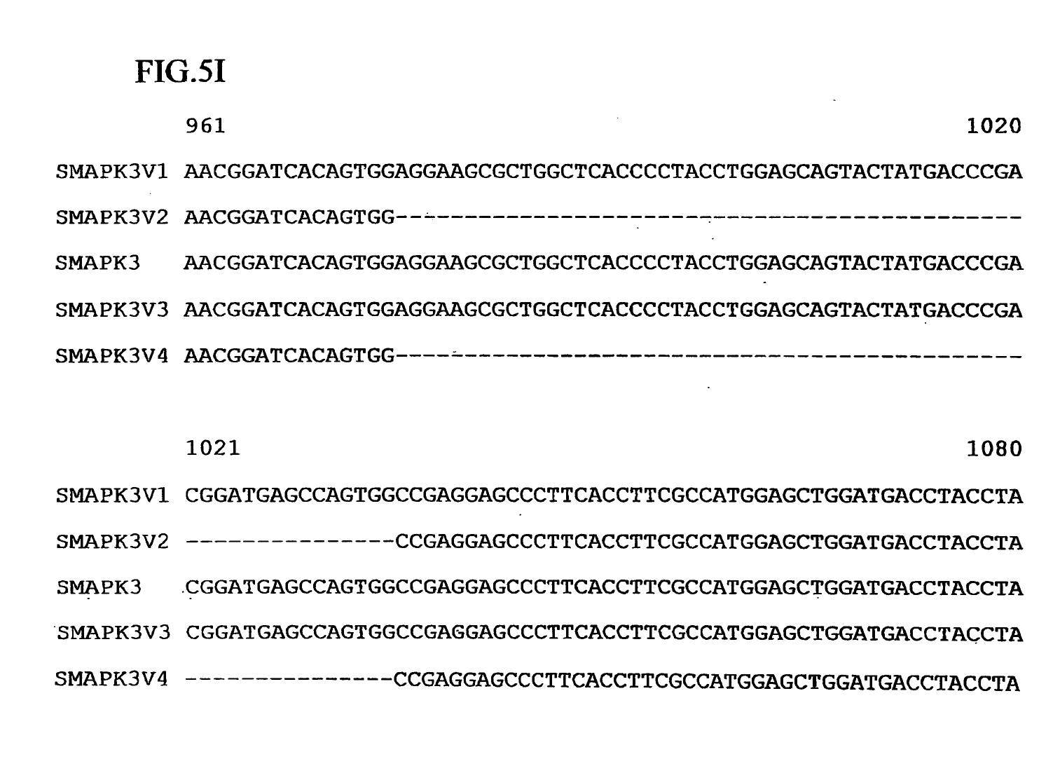

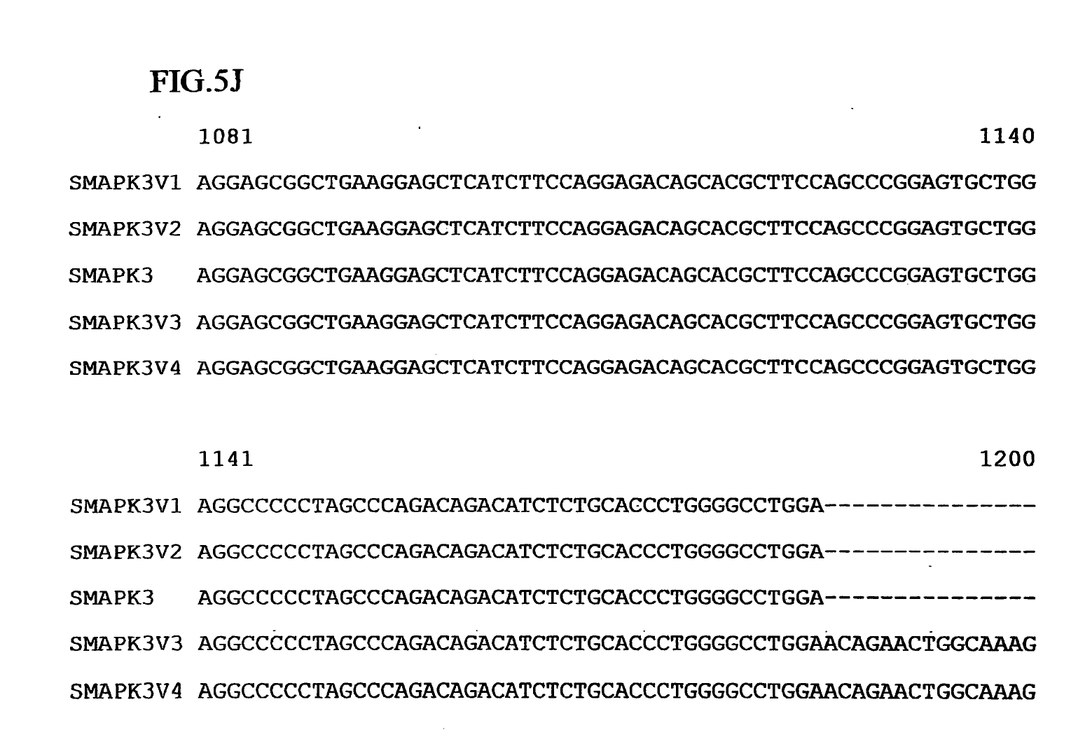

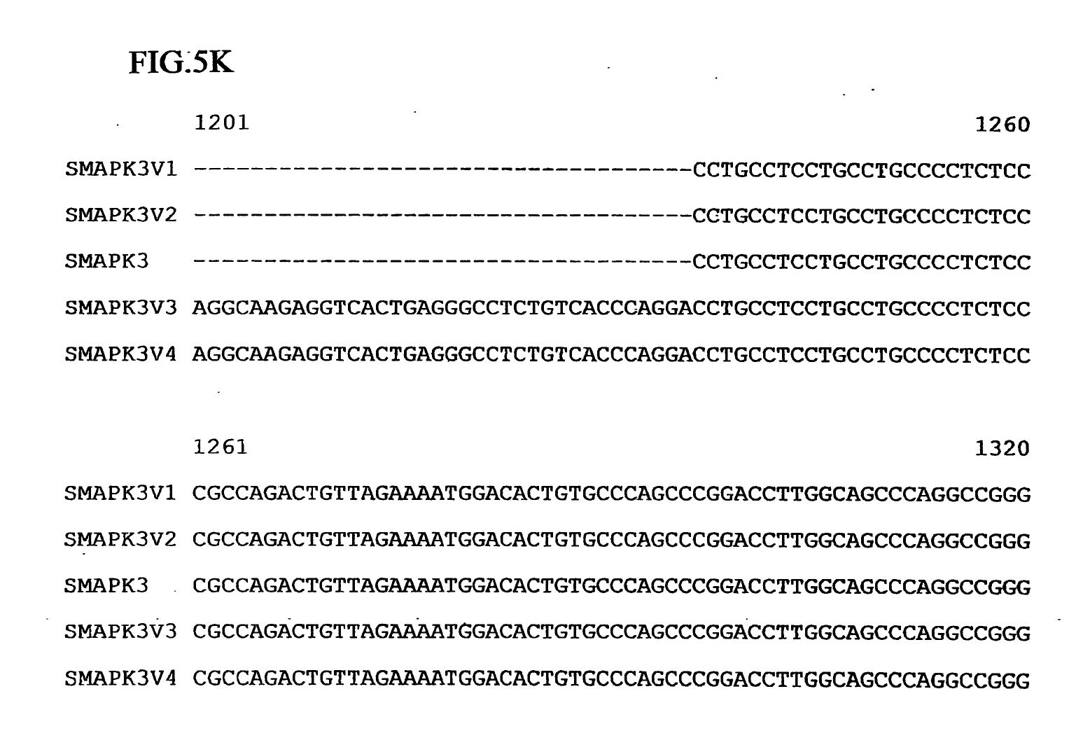

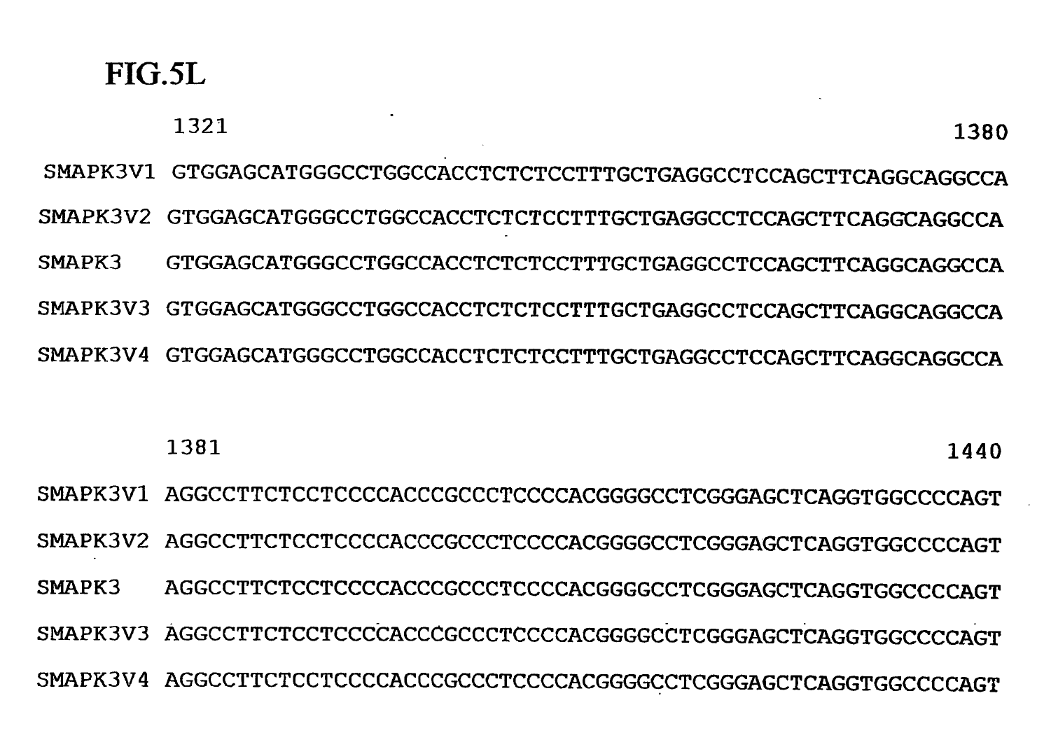

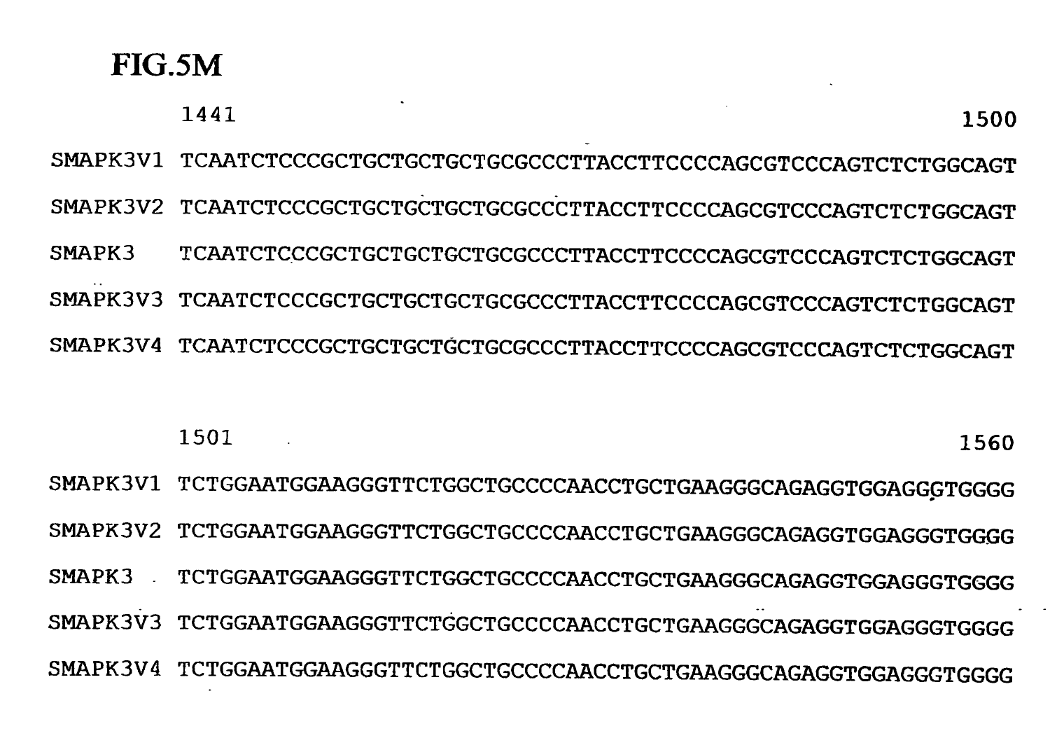

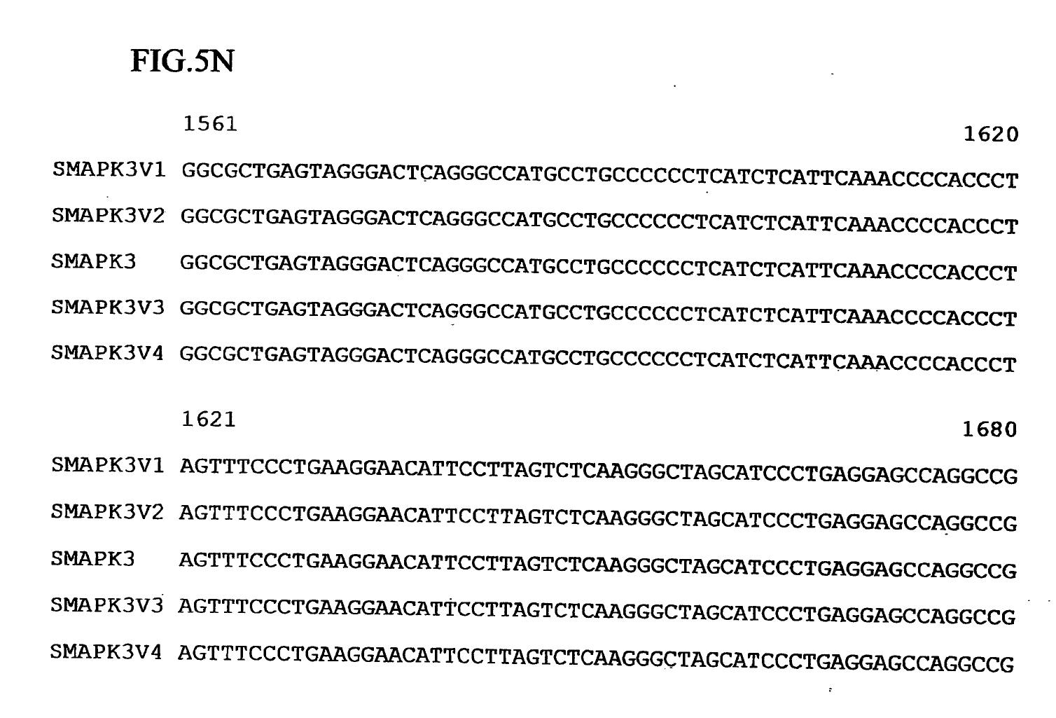

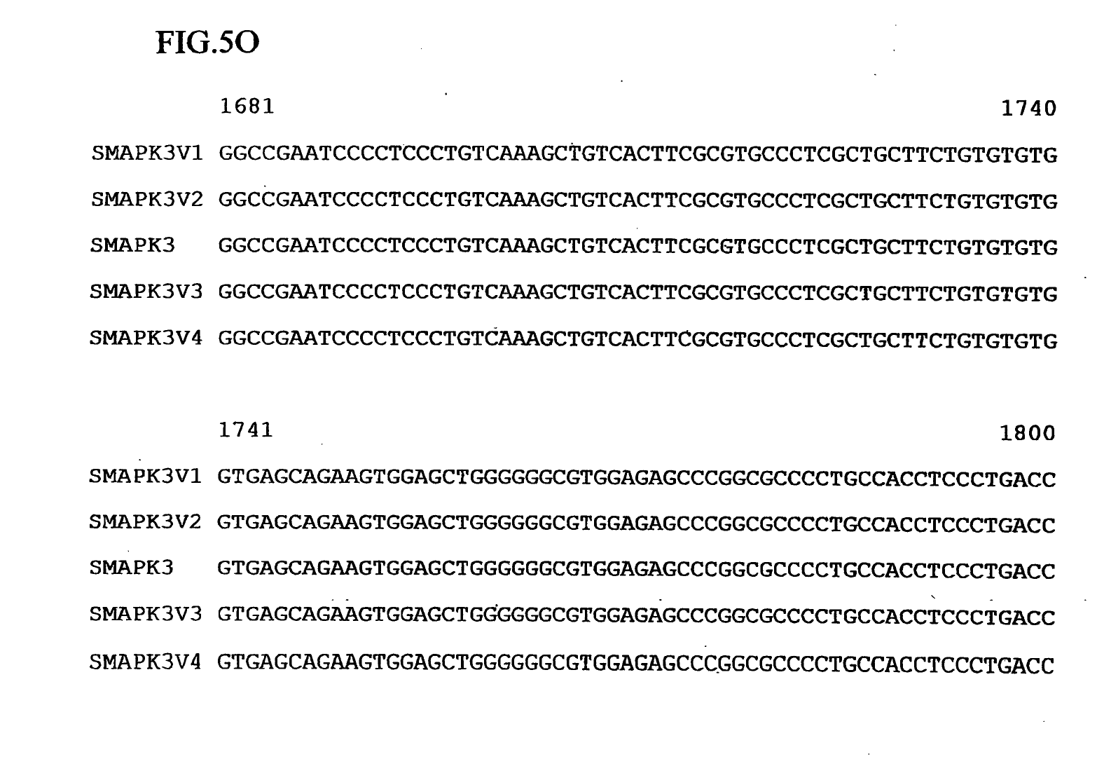

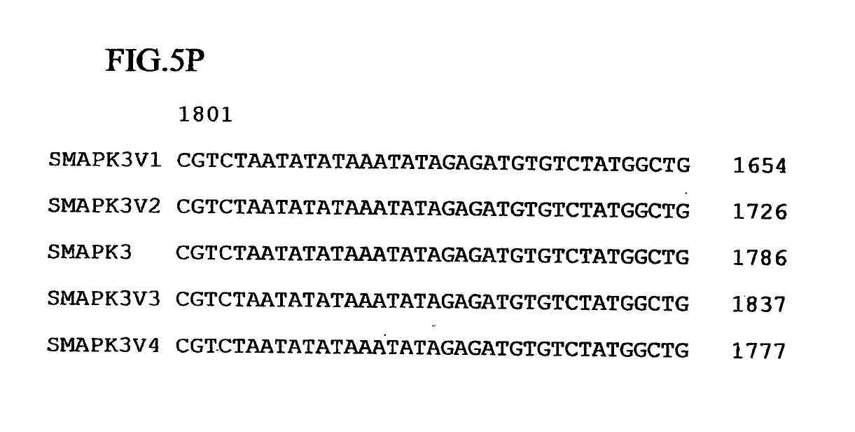

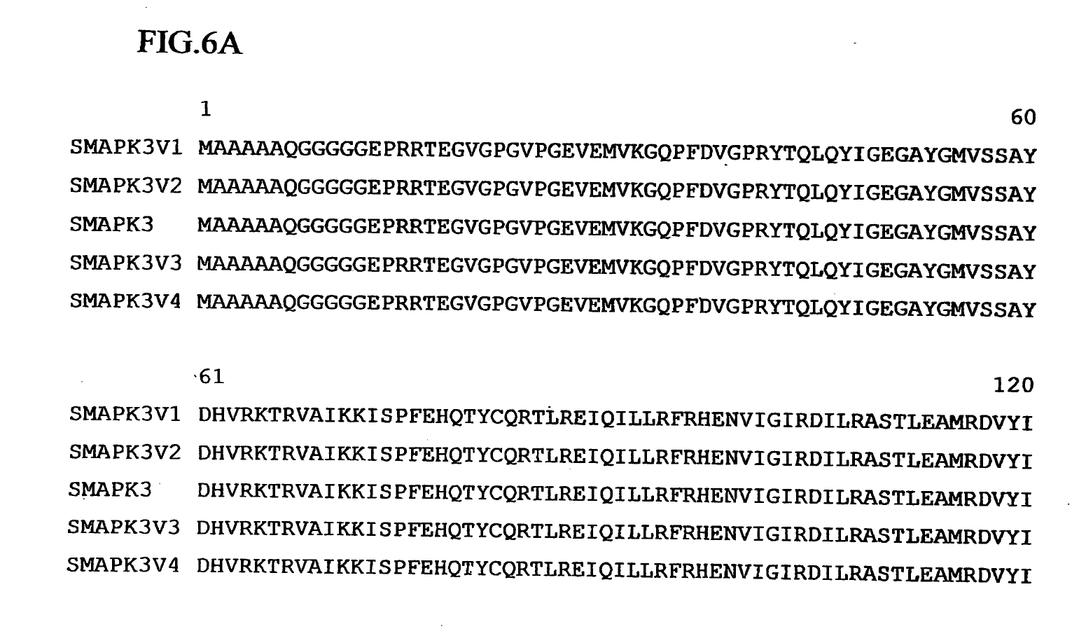

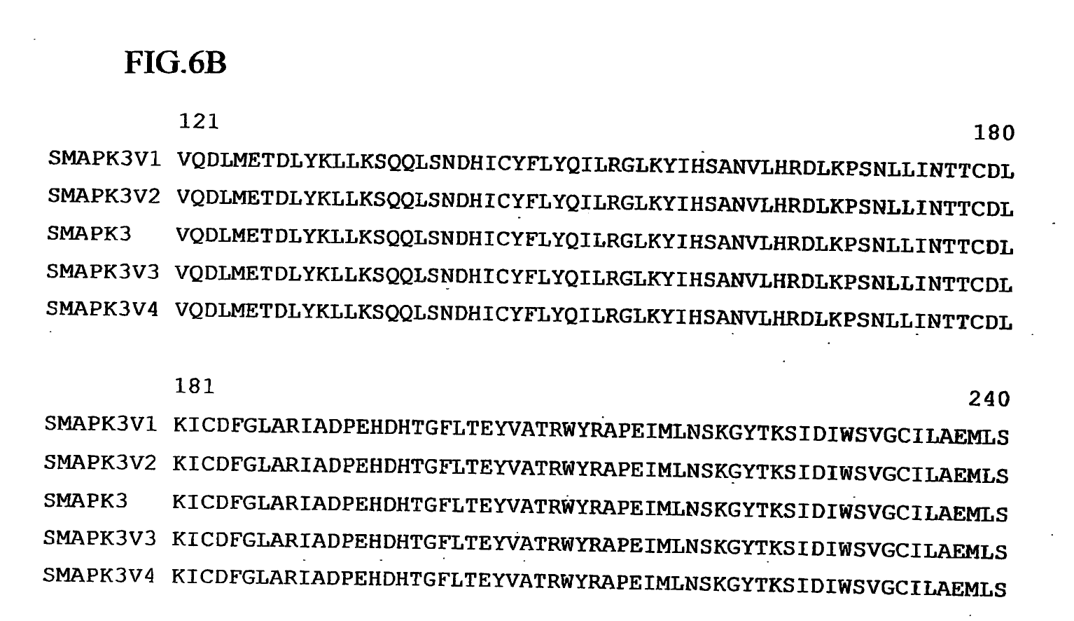

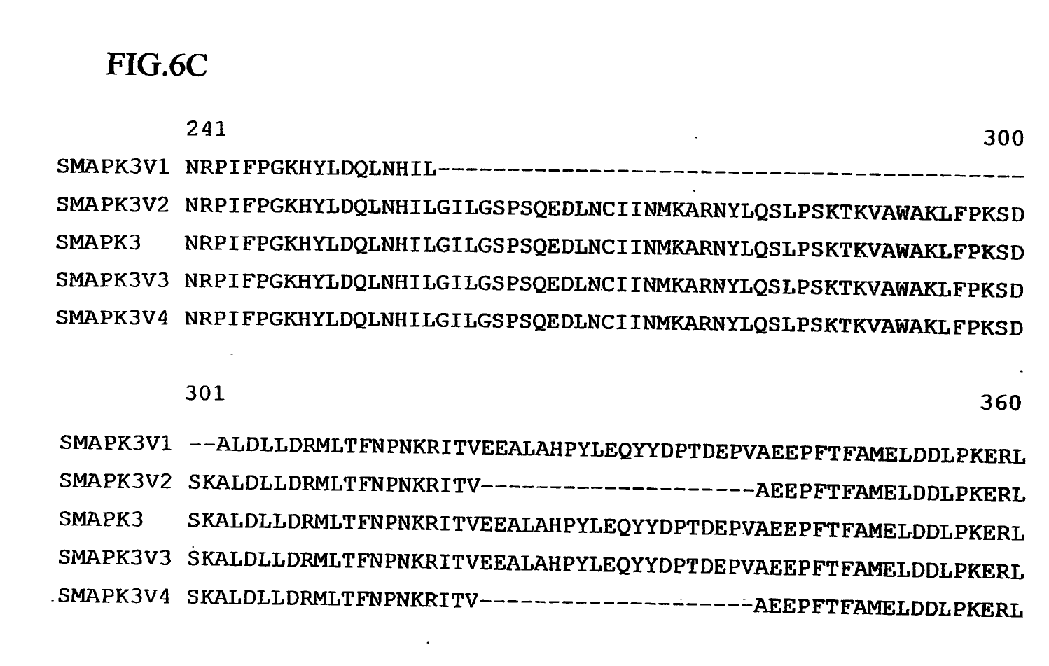

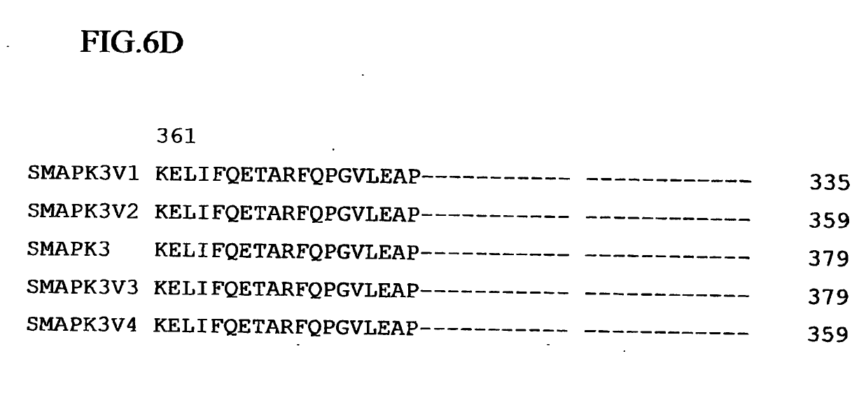

The full-length of the SMAPK3V1 cDNA is a 1654 bp clone containing a 1005 bp open reading frame (ORF) extending from nucleotides 12 to 1016, which corresponds to an encoded protein of 335 amino acid residues with a predicted molecular mass of 38.2 kDa. The full-length of the SMAPK3V2 cDNA is a 1726 bp clone containing a 1077 bp ORF extending from nucleotides 12 to 1088, which corresponds to an encoded protein of 359 amino acid residues with a predicted molecular mass of 40.8 kDa. The full-length of the SMAPK3V3 cDNA is a 1837 bp clone containing a 1137 bp ORF extending from nucleotides 12 to 1148, which corresponds to an encoded protein of 379 amino acid residues with a predicted molecular mass of 43.1 kDa. The full-length of the SMAPK3V4 cDNA is a 1777 bp clone containing a 1077 bp ORF extending from nucleotides 12 to 1088, which corresponds to an encoded protein of 359 amino acid residues with a predicted molecular mass of 40.8 kDa. The sequences around the initiation ATG codon of SMAPK3V1, SMAPK3V2, SMAPK3V3 and SMAPK3V4 (located at nucleotides 12 to 14) were similar to the Kozak consensus sequence (A/GCCATGG) (Kozak, (1987) Nucleic Acids Res. 15: 8125-48; Kozak, (1991) J Cell Biol. 115: 887-903.). To determine the variations (insertion/deletion) in sequences of SMAPK3V1, SMAPK3V2, SMAPK3V3 and SMAPK3V4 cDNA clones, an alignment of SMAPK3 nucleotide/amino acid sequence with these clones was performed (FIGS. 5 and 6). The results indicate that two genetic deletions and one genetic insertion were found in the aligned sequences. This information demonstrates that SMAPK3V1 is an in-frame 132 bp deletion (encoding 44 amino acids) in the sequence of SMAPK3 from nucleotides 787 to 918; SMAPK3V2 is an in-frame 60 bp deletion (encoding 20 amino acids) in the sequence of SMAPK3 from nucleotides 976 to 1035; and SMAPK3V3 is a 51 bp insertion at the 3′-untranslated region (3′-UTR) in the sequence of SMAPK3 from nucleotides 1185 to 1186; SMAPK3V4 is an in-frame 60 bp deletion (encoding 20 amino acids) in the sequence of SMAPK3 from nucleotides 976 to 1035 and a 51 bp insertion at the 3′-untranslated region (3′-UTR) in the sequence of SMAPK3 from nucleotides 1185 to 1186. Thus, SMAPK3V3 and SMAPK3 only differ in the nucleotide sequence but not in the amino acid sequence. The 51-nucleotide-insertion in the sequence of SMAPK3V3 is from nucleotides 1186 to 1236. SMAPK3V4 and SMAPK3V2 only differ in the nucleotide sequence but not in the amino acid sequence. The 51-nucleotide-insertion in the sequence of SMAPK3V4 is from nucleotides 1127to 1176.

In the invention, a search of ESTs deposited in dbEST (Boguski et al., (1993) Nat Genet. 4: 332-3) at NCBI was performed. Six ESTs were found to confirm the missing region described in SMAPK3V1, SMAPK3V2 and the inserted region described in SMAPK3V3. One EST (GenBank accession number BE879857), confirmed the absence of the 132 bp region in SMAPK3V 1 nucleotide sequence, was found to be isolated from a large cell lung cancer cDNA library. This suggests that the absence of the 132 bp nucleotide fragment located between nucleotides 786 and 787 of SMAPK3V1 may be a useful marker for large cell lung cancer diagnosis. One EST (GenBank accession number AL583197), confirmed the absence of the 60 bp region on SMAPK3V2 and SMAPK3V4 nucleotide sequences, was found to be isolated from a Burkitt lymphoma cDNA library. This suggests that the absence of the 60 bp nucleotide fragment located between nucleotides 975 and 976 of SMAPK3V2 may be a useful marker for Burkitt lymphoma diagnosis. Four ESTs (GenBank accession number BM041386, BM041252, BE891264, BE383357), confirmed the presence of the 51 bp region on SMAPK3V3 and SMAPK3V4 nucleotide sequences, were found to be isolated from many tumor cDNA library (e.g., kidney-Wilms' tumor, skin-melanotic melanoma, brain-neuroblastoma). This suggests that the presence of the 51 bp insertion fragment located between nucleotides 1185 and 1237 of SMAPK3V3 and nucleotides 1126 and 1177 of SMAPK3V4 is an important marker in association with cancers.

Therefore, any nucleotide fragments comprising nucleotides 783-788 (encoding amino acid residues 258-259) of SMAPK3V1, nucleotides 972-977 (encoding amino acid residue 321 to 322) of SMAPK3V2, nucleotides 1186-1236 of SMAPK3V3 or nucleotides 1127-1176 of SMAPK3V4 may be used as probes for determining the presence of the variants under high stringent conditions. An alternative approach is that any set of primers for amplifying the fragment containing nucleotides 783-788 of SMAPK3V1, nucleotides 972-977 of SMAPK3V2, nucleotides 1186-1236 of SMAPK3V3, and nucleotides 1127-1176 of SMAPK3V4 may be used for determining the presence of the variants.

According to the present invention, the polypeptides encoded by human SMAPK3-related gene variants (SMAPK3V1 and SMAPK3V2) and fragments thereof may be produced through genetic engineering techniques. In this case, they are produced by appropriate host cells that have been transformed by DNAs that code the polypeptides or fragments thereof. The nucleotide sequence encoding the polypeptide of the human SMAPK3-related gene variants or fragment thereof is inserted into an appropriate expression vector, i.e., a vector which contains the necessary elements for the transcription and translation of the inserted coding sequence in a suitable host. The nucleic acid sequence is inserted into the vector in a manner that it will be expressed under appropriate conditions (e.g., in proper orientation and correct reading frame and with appropriate expression sequences, including an RNA polymerase binding sequence and a ribosomal binding sequence).

Any method that is known to those skilled in the art may be used to construct expression vectors containing the sequences encoding the polypeptides of the human SMAPK3-related gene variants and appropriate transcriptional/translational control elements. These methods may include in vitro recombinant DNA and synthetic techniques, and in vivo genetic recombinants. (See, e.g., Sambrook, J. Cold Spring Harbor Press, Plainview N.Y., ch. 4, 8, and 16-17; Ausubel, R. M. et al. (1995) Current protocols in Molecular Biology, John Wiley & Sons, New York N.Y., ch. 9, 13, and 16.)

A variety of expression vector/host systems may be utilized to express the polypeptide-coding sequence. These include, but are not limited to, microorganisms such as bacteria transformed with recombinant bacteriophage, plasmid, or cosmid DNA expression vector; yeast transformed with yeast expression vector; insect cell systems infected with virus (e.g., baculovirus); plant cell system transformed with viral expression vector (e.g., cauliflower mosaic virus, CaMV, or tobacco mosaic virus, TMV); or animal cell system infected with virus (e.g., vaccina virus, adenovirus, etc.). Preferably, the host cell is a bacterium, and most preferably, the bacterium is E. coli.

Alternatively, the polypeptides encoded by human SMAPK3-related gene variants or fragments thereof may be synthesized using chemical methods. For example, peptide synthesis can be performed using various solid-phase techniques (Roberge, J. Y. et al. (1995) Science 269: 202 to 204). Automated synthesis may be achieved using the ABI 431A peptide synthesizer (Perkin-Elmer).

According to the present invention, the fragments of the polypeptides and nucleic acid sequences of the human SMAPK3-related gene variants are used as immunogens and primers or probes, respectively. It is preferable to use the purified fragments of the human SMAPK3-related gene variants. The fragments may be produced by enzyme digestion, chemical cleavage of isolated or purified polypeptide or nucleic acid sequences, or chemical synthesis and then may be isolated or purified. Such isolated or purified fragments of the polypeptides and nucleic acid sequences can be used directly as immunogens and primers or probes, respectively.

The present invention further provides the antibodies which specifically bind one or more out-surface epitopes of the polypeptides encoded by human SMAPK3-related gene variants.

According to the present invention, immunization of mammals with immunogens described herin, preferably humans, rabbits, rats, mice, sheep, goats, cows, or horses, is performed following procedures well known to those skilled in the art, for the purpose of obtaining antisera containing polyclonal antibodies or hybridoma lines secreting monoclonal antibodies.

Monoclonal antibodies can be prepared by standard techniques, given the teachings contained herein. Such techniques are disclosed, for example, in U.S. Pat. No. 4,271,145 and U.S. Pat. No. 4,196,265. Briefly, an animal is immunized with the immunogen. Hybridomas are prepared by fusing spleen cells from the immunized animal with myeloma cells. The fusion products are screened for those producing antibodies that bind to the immunogen. The positive hybridoma clones are isolated, and the monoclonal antibodies are recovered from those clones.

Immunization regimens for production of both polyclonal and monoclonal antibodies are well-known in the art. The immunogen may be injected by any of a number of routes, including subcutaneous, intravenous, intraperitoneal, intradermal, intramuscular, mucosal, or a combination thereof. The immunogen may be injected in soluble form, aggregate form, attached to a physical carrier, or mixed with an adjuvant, using methods and materials well-known in the art. The antisera and antibodies may be purified using column chromatography methods well known to those skilled in the art.

According to the present invention, antibody fragments which contain specific binding sites for the polypeptides or fragments thereof may also be generated. For example, such fragments include, but are not limited to, F(ab′)₂ fragments produced by pepsin digestion of the antibody molecule and Fab fragments generated by reducing the disulfide bridges of the F(ab′)₂ fragments.

Many gene variants have been found to be associated with diseases (Stallings-Mann et al., (1996) Proc Natl Acad Sci U S A 93: 12394-9; Liu et al., (1997) Nat Genet 16:328-9; Siffert et al., (1998) Nat Genet 18: 45 to 8; Lukas et al., (2001) Cancer Res 61: 3212 to 9). Based on the cDNA libraries of the matched ESTs, SMAPK3V1 can be specifically associated with NSCLC, SMAPK3V2 can be associated with Burkitt lymphoma whereas SMAPK3V3 and SMAPK3V4 can be associated with general cancers. Thus, the expression level of SMAPK3V1, SMAPK3V2, SMAPK3V3 and SMAPK3V4 each relative to SMAPK3 may be a useful indicator for screening of patients suspected of having cancers, or more specifically, the NSCLC or lymphoma. This suggests that the index of relative expression level (mRNA or protein) may associate with an increased susceptibility to cancers or NSCLC, more preferably, large cell lung cancer or Burkitt lymphoma. Fragments of SMAPK3V1, SMAPK3V2, SMAPK3V3 and SMAPK3V4 transcripts (mRNAs) may be detected by RT-PCR approach. Polypeptides encoded by the SMAPK3-related gene variants may be determined by the binding of antibodies to these polypeptides. These approaches may be performed in accordance with conventional methods well known by persons skilled in the art.

The subject invention also provides methods for diagnosing the diseases associated with the deficiency of human SMAPK3 gene in a mammal, in particular, homeostasis impairment-related diseases and non-small cell lung cancer, e.g. large cell lung cancer and Burkitt lymphoma.

The method for diagnosing the diseases associated with the deficiency of human SMAPK3 gene may be performed by detecting the nucleotide sequences of SMAPK3V1, SMAPK3V2, SMAPK3V3 or SMAPK3V4 of the invention, which comprises the steps of: (1) extracting total RNA of cells obtained from a mammal; (2) amplifying the RNA by reverse transcriptase-polymerase chain reaction (RT-PCR) with a set of primers to obtain a cDNA comprising the fragments comprising nucleotides 783-788 of SEQ ID NO: 1 or nucleotides 972-977 of SEQ ID NO: 3 or nucleotides located between nucleotides 1186-1236 of SEQ ID NO: 5 or nucleotides located between nucleotides 1127-1176 of SEQ ID NO: 7; and (3) detecting whether the cDNA sample is obtained. If necessary, the amount of the obtained cDNA sample may be detected.

In this embodiment, a forward primer may be designed to have a sequence comprising nucleotides 783-788 of SEQ ID NO: 1 and a reverse primer may be designed to have a sequence complementary to the nucleotides of SEQ ID NO: 1 at any other locations downstream of nucleotide 788; or a forward primer has a sequence comprising nucleotides 972-977 of SEQ ID NO: 3/SEQ ID NO: 7 and a reverse primer has a sequence complementary to the nucleotides of SEQ ID NO: 3/SEQ ID NO: 7 at any other locations downstream of nucleotide 977; or a forward primer has a sequence comprising nucleotides 1186-1236 of SEQ ID NO: 5 and a reverse primer has a sequence complementary to the nucleotides of SEQ ID NO: 5 at any other locations downstream of nucleotide 1236; or a forward primer has a sequence comprising nucleotides 1127-1176 of SEQ ID NO: 7 and a reverse primer has a sequence complementary to the nucleotides of SEQ ID NO: 7 at any other locations downstream of nucleotide 1176. Alternatively, the reverse primer may be designed to have a sequence complementary to the nucleotides of SEQ ID NO: 1 containing nucleotides 783-788 and the forward primer may be designed to have a sequence comprising the nucleotides of SEQ ID NO: 1 at any other locations upstream of nucleotide 783; or the reverse primer has a sequence complementary to the nucleotides of SEQ ID NO: 3/SEQ ID NO: 7 containing nucleotides 972-977 and the forward primer has a sequence comprising the nucleotides of SEQ ID NO: 3/SEQ ID NO: 7 at any other locations upstream of nucleotide 972; or the reverse primer has a sequence complementary to the nucleotides of SEQ ID NO: 5/SEQ ID NO: 7 containing nucleotides between 1186-1236/1127-1176 and the forward primer has a sequence comprising the nucleotides of SEQ ID NO: 5/SEQ ID NO: 7 at any other locations upstream of nucleotide 1186/1127. In this case, only SMAPK3V1, SMAPK3V2, SMAPK3V3 and SMAPK3V4 will be amplified. Preferably, the primers of the invention contain 15 to 30 nucleotides.

Alternatively, the forward primer may be designed to have a sequence comprising the nucleotides of SEQ ID NO: 1 at any locations upstream of nucleotide 783 and the reverse primer may be designed to have a sequence complementary to the nucleotides of SEQ ID NO: 1 at any other locations downstream of nucleotide 788; or the forward primer has a sequence comprising the nucleotides of SEQ ID NO: 3 at any locations upstream of nucleotide 972 and the reverse primer has a sequence complementary to the nucleotides of SEQ ID NO: 3 at any other locations downstream of nucleotide 977; or the forward primer has a sequence comprising the nucleotides of SEQ ID NO: 5 at any locations upstream of nucleotide 1186 and the reverse primer has a sequence complementary to the nucleotides of SEQ ID NO: 5 at any other locations downstream of nucleotide 1236; or the forward primer has a sequence comprising the nucleotides of SEQ ID NO: 7 at any locations upstream of nucleotide 1127 and the reverse primer has a sequence complementary to the nucleotides of SEQ ID NO: 7 at any other locations downstream of nucleotide 1177. In this case, SMAPK3V1, SMAPK3V2, SMAPK3V3 or SMAPK3V4 together with SMAPK3 in a sample will be amplified. The length of the PCR fragment from SMAPK3V1 will be 132 bp shorter than that from SMAPK3; the length of the PCR fragment from SMAPK3V2 will be 60 bp shorter than that from SMAPK3; the length of the PCR fragment from SMAPK3V3 will be 51 bp longer than that from SMAPK3; and the length of the PCR fragment from SMAPK3V4 will be 9 bp shorter than that from SMAPK3.

Preferably, the primers of the invention contain 15 to 30 nucleotides.

Total RNA may be isolated from patient samples by using TRIZOL reagents (Life Technology). Tissue samples (e.g., biopsy samples) are powdered under liquid nitrogen before homogenization. RNA purity and integrity are assessed by absorbance at 260/280 nm and by agarose gel electrophoresis. The set of primers designed to amplify the expected sizes of specific PCR fragments of gene variants (SMAPK3V1, SMAPK3V2, SMAPK3V3 and SMAPK3V4) can be used. PCR fragments are analyzed on a 1% agarose gel using five microliters (10%) of the amplified products. The intensity of the signals may be determined by using the Molecular Analyst program (version 1.4.1; Bio-Rad). Thus, the index of relative expression levels for each co-amplified PCR products may be calculated based on the intensity of signals.

The RT-PCR experiment may be performed according to the manufacturer instructions (Boehringer Mannheim). A 50 μl reaction mixture containing 2 μl total RNA (0.1 μg/μl), 1 μl each primer (20 pM), 1 μl each dNTP (10 mM), 2.5 μl DTT solution (100 mM), 10 μl 5×RT-PCR buffer, 1 μl enzyme mixture, and 28.5 μl sterile distilled water may be subjected to the conditions such as reverse transcription at 60° C. for 30 minutes followed by 35 cycles of denaturation at 94° C. for 2 minutes, annealing at 60° C. for 2 minutes, and extension at 68° C. for 2 minutes. The RT-PCR analysis may be repeated twice to ensure reproducibility, for a total of three independent experiments.

Another embodiment of the method for diagnosing the diseases associated with the deficiency of human SMAPK3 gene is performed by detecting the nucleotide sequence of SMAPK3V1, SMAPK3V2, SMAPK3V3 or SMAPK3V4, which comprises the steps of: (1) extracting total RNA from a sample obtained from the mammal; (2) amplifying the RNA by reverse transcriptase-polymerase chain reaction (RT-PCR) to obtain a cDNA sample; (3) bringing the cDNA sample into contact with the nucleic acid selected from the group consisting of SEQ ID NOs: 1, 3 and 5, and the fragments thereof; and (4) detecting whether the cDNA sample hybridizes with the nucleic acid of SEQ ID NOs: 1, 3, 5 or 7, or the fragments thereof. If necessary, the amount of hybridized sample may be detected.

The expression of gene variants can be analyzed using Northern Blot hybridization approach. Specific fragment which comprising nucleotides 783-788 of the SMAPK3V1, nucleotides 972-977 of the SMAPK3V2/SMAPK3V4, nucleotides 1186-1236 of the SMAPK3V3 or nucleotides 1127-1176 of the SMAPK3V4 may be amplified by polymerase chain reaction (PCR) using primer set designed for RT-PCR. The amplified PCR fragment may be labeled and serve as a probe to hybridize the membranes containing total RNAs extracted from the samples under the conditions of 55° C. in a suitable hybridization solution for 3 hours. Blots may be washed twice in 2×SSC, 0.1% SDS at room temperature for 15 minutes each, followed by two washes in 0.1×SSC and 0.1% SDS at 65° C. for 20 minutes each. After these washes, blot may be rinsed briefly in suitable washing buffer and incubated in blocking solution for 30 minutes, and then incubated in suitable antibody solution for 30 minutes. Blots may be washed in washing buffer for 30 minutes and equilibrated in suitable detection buffer before detecting the signals. Alternatively, the presence of gene variants (cDNAs or PCR) can be detected using microarray approach. The cDNAs or PCR products corresponding to the nucleotide sequences of the present invention may be immobilized on a suitable substrate such as a glass slide. Hybridization can be preformed using the labeled mRNAs extracted from samples. After hybridization, nonhybridized mRNAs are removed. The relative abundance of each labeled transcript, hybridizing to a cDNA/PCR product immobilized on the microarray, can be determined by analyzing the scanned images.

According to the present invention, the method for diagnosing the diseases associated with the deficiency of human SMAPK3 gene may also be performed by detecting the polypeptides encoded by SMAPK3V1 and SMAPK3V2 of the invention. For instance, the polypeptides in protein samples obtained from the mammal may be determined by, but is not limited to, the immunoassay wherein the antibody specifically binding to the polypeptides of the invention is contacted with the protein sample, and the antibody-polypeptide complex is detected. If necessary, the amount of the antibody-polypeptide complexes can be determined.

The polypeptides encoded by the gene variants may be expressed in prokaryotic cells by using suitable prokaryotic expression vectors. The cDNA fragments of SMAPK3V1 or SMAPK3V2 genes encoding the amino acid coding sequence may be PCR amplified with restriction enzyme digestion sites incorporated in the 5′ and 3′ ends, respectively. The PCR products can then be enzyme digested, purified, and inserted into the corresponding sites of prokaryotic expression vector in-frame to generate recombinant plasmids. Sequence fidelity of this recombinant DNA can be verified by sequencing. The prokaryotic recombinant plasmids may be transformed into host cells (e.g., E. coli BL21 (DE3)). Recombinant protein synthesis may be stimulated by the addition of 0.4 mM isopropylthiogalactoside (IPTG) for 3 hours. The bacterially-expressed proteins may be purified.

The polypeptides encoded by SMAPK3-related gene variants may be expressed in animal cells by using eukaryotic expression vectors. Cells may be maintained in Dulbecco's modified Eagle's medium (DMEM) supplemented with 10% fetal bovine serum (FBS; Gibco BRL) at 37° C. in a humidified 5% CO₂ atmosphere. Before transfection, the nucleotide sequence of each of the gene variant may be amplified with PCR primers containing restriction enzyme digestion sites and ligated into the corresponding sites of eukaryotic expression vector in-frame. Sequence fidelity of this recombinant DNA can be verified by sequencing. The cells may be plated in 12-well plates one day before transfection at a density of 5×10⁴ cells per well. Transfections may be carried out using Lipofectamine Plus transfection reagent according to the manufacturer's instructions (Gibco BRL). Three hours following transfection, medium containing the complexes may be replaced with fresh medium. Forty-eight hours after incubation, the cells may be scraped into lysis buffer (0.1 M Tris HCl, pH 8.0, 0.1% Triton X-100) for purification of expressed proteins. After these proteins are purified, monoclonal antibodies against these purified proteins (SMAPK3V1 and SMAPK3V2). may be generated using hybridoma technique according to the conventional methods (de StGroth and Scheidegger, (1980) J Immunol Methods 35:1-21; Cote et al. (1983) Proc Natl Acad Sci U S A 80: 2026-30; and Kozbor et al. (1985) J Immunol Methods 81:31-42).

According to the present invention, the presence of the polypeptides encoded by the gene variants in samples of normal lung and lung cancers may be determined by, but is not limited to, Western blot analysis. Proteins extracted from samples may be separated by SDS-PAGE and transferred to suitable membranes such as polyvinylidene difluoride (PVDF) in transfer buffer (25 mM Tris-HCl, pH 8.3, 192 mM glycine, 20% methanol) with a Trans-Blot apparatus for 1 hour at 100 V (e.g., Bio-Rad). The proteins can be immunoblotted with specific antibodies. For example, membrane blotted with extracted proteins may be blocked with suitable buffers such as 3% solution of BSA or 3% solution of nonfat milk powder in TBST buffer (10 mM Tris-HCl, pH 8.0, 150 mM NaCl, 0.1% Tween 20) and incubated with monoclonal antibody directed against the polypeptides encoded by the gene variants. Unbound antibody is removed by washing with TBST for 5×1 minutes. Bound antibody may be detected using commercial ECL Western blotting detecting reagents.

The following examples are provided for illustration, but not for limiting the invention.

### EXAMPLES

### Analysis of Human Lung EST Databases

Expressed sequence tags (ESTs) generated from the large-scale PCR-based sequencing of the 5′-end of human clones from many cDNA libraries (a normal lung, a large cell lung cancer, a squamous cell lung cancer, a small cell lung cancer, a Burkitt lymphoma, and a pooled cancer tissues) were compiled and served as EST databases. Sequence comparisons against the nonredundant nucleotide and protein databases were performed using BLASTN and BLASTX programs (Altschul et al., (1997) Nucleic Acids Res. 25: 3389-3402; Gish and States, (1993) Nat Genet 3:266-272), at the National Center for Biotechnology Information (NCBI) with a significance cutoff of p<10⁻¹⁰. ESTs representing putative SMAPK3 encoding gene were identified during the course of EST generation.

### Isolation of cDNA Clones

Four cDNA clones exhibiting EST sequences similar to the SMAPK3 gene were isolated from the lung and pooled pooled cancer cDNA libraries and named SMAPK3V1, SMAPK3V2, SMAPK3V3 and SMAPK3V4. The inserts of these clones were subsequently excised in vivo from the λZAP Express vector using the ExAssist/XLOLR helper phage system (Stratagene). Phagemid particles were excised by coinfecting XL1-BLUE MRF′ cells with ExAssist helper phage. The excised pBluescript phagemids were used to infect E. coli XLOLR cells, which lack the amber suppressor necessary for ExAssist phage replication. Infected XLOLR cells were selected using kanamycin resistance. Resultant colonies contained the double stranded phagemid vector with the cloned cDNA insert. A single colony was grown overnight in LB-kanamycin, and DNA was purified using a Qiagen plasmid purification kit.

### Full Length Nucleotide Sequencing and Database Comparisons

Phagemid DNA was sequenced using the Epicentre#SE9101LC SequiTherm EXCEL™II DNA Sequencing Kit for 4200S-2 Global NEW IR² DNA sequencing system (LI-COR). Using the primer-walking approach, full-length sequence was determined. Nucleotide and protein searches were performed using BLAST against the non-redundant database of NCBI.

### In Silico Tissue Distribution Analysis

The coding sequence for each cDNA clones was searched against the dbEST sequence database (Boguski et al., (1993) Nat Genet. 4: 332-3) using the BLAST algorithm at the NCBI website. ESTs derived from each tissue were used as a source of information for transcript tissue expression analysis. Tissue distribution for each isolated cDNA clone was determined by ESTs matching to that particular sequence variants (insertions or deletions) with a significance cutoff of p<10⁻¹⁰.

### References

Altschul et al., Gapped BLAST and PSI-BLAST: a new generation of protein database search programs, Nucleic Acids Res, 25: 3389-3402, (1997).
Ausubel et al., Current protocols in Molecular Biology, John Wiley & Sons, New York N.Y., ch. 9, 13, and 16, (1995).
Bauer et al., Role of p42/p44 mitogen-activated-protein kinase and p21 waf1/cip 1 in the regulation of vascular smooth muscle cell proliferation by nitric oxide. Proc Natl Acad Sci U S A 98:12802-7, (2001).
Blenis, J. Signal transduction via the MAP kinases: proceed at your own RSK. Proc Natl Acad Sci U S A 90:5889-92, (1993).
Boguski et al., dbEST—database for “expressed sequence tags”. Nat Genet. 4: 332-3, (1993).
Carney, D. N. The biology of lung cancer. Curr. Opin. Oncol. 4: 292-8, (1992a).
Carney, D. N. Biology of small-cell lung cancer. Lancet 339: 843-6, (1992b).
Cote et al., Generation of human monoclonal antibodies reactive with cellular antigens, Proc Natl Acad Sci U S A 80: 2026-30 (1983).
Delmas et al., The p42/p44 mitogen-activated protein kinase activation triggers p27Kip1 degradation independently of CDK2/cyclin E in NIH 3T3 cells. J Biol Chem 276:34958-65, (2001).
de StGroth and Scheidegger, Production of monoclonal antibodies: strategy and tactics, J Immunol Methods 35:1-21, (1980).
Genoscope EST Accession No. AL583197
Gish and States, Identification of protein coding regions by database similarity search, Nat Genet, 3:266-272, (1993).
Ihde and Minna, Non-small cell lung cancer. Part II: Treatment. Curr. Probl. Cancer 15: 105-54, (1991).
Kozak, An analysis of 5′-noncoding sequences from 699 vertebrate messenger RNAs. Nucleic Acids Res, 15: 8125-48, (1987).
Kozak, An analysis of vertebrate mRNA sequences: intimations of translational control, J Cell Biol, 115: 887-903, (1991).
Kozbor et al., Specific immunoglobulin production and enhanced tumorigenicity following ascites growth of human hybridomas, J Immunol Methods, 81:31-42 (1985).
Le Gall et al., The p42/p44 MAP kinase pathway prevents apoptosis induced by anchorage and serum removal. Mol Biol Cell 11: 1103-12, (2000).
Liu et al., Silent mutation induces exon skipping of fibrillin-1 gene in Marfan syndrome. Nat Genet 16:328-9, (1997).
Lukas et al., Alternative and aberrant messenger RNA splicing of the mdm2 oncogene in invasive breast cancer. Cancer Res 61:3212-9, (2001).
Roberge et al., A strategy for a convergent synthesis of N-linked glycopeptides on a solid support. Science 269:202-4, (1995).
Sambrook, J. Cold Spring Harbor Press, Plainview N.Y., ch. 4, 8, and 16-17.
Sethi, Science, medicine, and the future. Lung cancer, BMJ, 314: 652-655, (1997)
Siffert et al., Association of a human G-protein beta3 subunit variant with hypertension. Nat Genet, 18:45-8, (1998).
Smyth et al., The impact of chemotherapy on small cell carcinoma of the bronchus. Q J Med, 61: 969-76, (1986).
Stallings-Mann et al., Alternative splicing of exon 3 of the human growth hormone receptor is the result of an unusual genetic polymorphism. Proc Natl Acad Sci U S A 93:12394-9, (1996).
Strausberg, R. EST Accession No. BE879857, BM041386, BM041252, BE891264, BE383357.
Strausberg, R. Protein Accession No. AAH 13992.

1. An isolated polypeptide comprising an amino acid sequence selected from the group consisting of SEQ ID NOS: 2 and 4, and fragments thereof:

2. The isolated polypeptide of claim 1, wherein the fragment comprises the amino acid residues 258 to 259 of SEQ ID NO: 2.

3. The isolated polypeptide of claim 1, wherein the fragment comprises the amino acid residues 321 to 322 of SEQ ID NO: 4.

4. An isolated nucleic acid comprising a nucleotide sequence selected from the group consisting of SEQ ID NOS: 1, 3, 5 and 7, and fragments thereof.

5. The isolated nucleic acid of claim 4, wherein the fragment comprises nucleotides 783 to 788 of SEQ ID NO: 1.

6. The isolated nucleic acid of claim 4, wherein the fragment comprises nucleotides 972 to 977 of SEQ ID NO: 3.

7. The isolated nucleic acid of claim 4, wherein the fragment comprises nucleotides 1186 to 1236 of SEQ ID NO: 5.

8. The isolated nucleic acid of claim 4, wherein the fragment comprises nucleotides 1127 to 1176 of SEQ ID NO: 7.

9. An expression vector comprising the nucleic acid of claim 4.

10. A host cell transformed with the expression vector of claim 9.

11. A method for producing an isolated polypeptide comprising an amino acid sequence selected from the group consisting of SEQ ID NOS: 2 and 4, and fragments thereof, which comprises the steps of:
(1) culturing the host cell of claim 10 under a condition suitable for the expression of the polypeptide; and
(2) recovering the polypeptide from the host cell culture.

12. An antibody specifically binding to the polypeptide of claim 1.

13. A method for diagnosing the diseases associated with the deficiency of the SMAPK3 gene in a mammal, in particular cancers, which comprises detecting the nucleic acid of claim 4.

14. The method of claim 13, wherein the detection of the nucleic acid of comprises the steps of:
(1) extracting total RNA from a sample obtained from the mammal;
(2) amplifying the RNA by reverse transcriptase-polymerase chain reaction (RT-PCR) to obtain a cDNA sample;
(3) bringing the cDNA sample into contact with the nucleic acid; and
(4) detecting whether the cDNA hybridizes with the nucleic acid;

15. The method of claim 14 further comprising the step of determining the amount of hybridized sample.

16. The method of claim 13, wherein the detection of the nucleic acid comprises the steps of:
(1) extracting the total RNAs of cells obtained from the mammal;
(2) amplifying the RNA by reverse transcriptase-polymerase chain reaction (RT-PCR) with a set of primers to obtain a cDNA comprising the fragment comprising the nucleotides 783 to 788 of SEQ ID NO: 1, the nucleotides 972 to 977 of SEQ ID NO: 3, the nucleotides 1186 to 1236 of SEQ ID NO: 5, or the nucleotides 1127 to 1176 of SEQ ID NO: 7; and
(3) detecting whether the cDNA is obtained.

17. The method of claim 16, wherein the forward primer has a sequence comprising the nucleotides 783 to 788 of SEQ ID NO: 1 and the reverse primer has a sequence complementary to the nucleotides of SEQ ID NO: 1 at any other locations downstream of nucleotide 788, or alternatively, the reverse primer has a sequence complementary to the nucleotides comprising the nucleotides 783 to 788 of SEQ ID NO: 1 and the forward primer has a sequence comprising the nucleotides of SEQ ID NO: 1 at any other locations upstream of nucleotide 783.

18. The method of claim 16, wherein the forward primer has a sequence comprising the nucleotides 972 to 977 of SEQ ID NO: 3 and the reverse primer has a sequence complementary to the nucleotides of SEQ ID NO: 3 at any other locations downstream of nucleotide 977, or alternatively, the reverse primer has a sequence complementary to the nucleotides comprising the nucleotides 972 to 977 of SEQ ID NO: 3 and the forward primer has a sequence comprising the nucleotides of SEQ ID NO: 3 at any other locations upstream of nucleotide 972.

19. The method of claim 16, wherein the forward primer has a sequence comprising the nucleotides between 1186 to 1236 of SEQ ID NO: 5 and the reverse primer has a sequence complementary to the nucleotides of SEQ ID NO: 5 at any other locations downstream of nucleotide 1236, or alternatively, the reverse primer has a sequence complementary to the nucleotides 1186 to 1236 of SEQ ID NO: 5 and the forward primer has a sequence comprising the nucleotides of SEQ ID NO: 5 at any other locations upstream of nucleotide 1186.

20. The method of claim 16, wherein the forward primer has a sequence comprising the nucleotides between 972 to 977 of SEQ ID NO: 7 and the reverse primer has a sequence complementary to the nucleotides comprising the nucleotide 1127 to 1176 of SEQ ID NO: 7.

21. The method of claim 16, wherein the forward primer has a sequence comprising the nucleotides of SEQ ID NO: 1 at any other locations upstream of nucleotide 783 and the reverse primer has a sequence complementary to the nucleotides of SEQ ID NO: 1 at any other locations downstream of nucleotide 788.

22. The method of claim 16, wherein the forward primer has a sequence comprising the nucleotides of SEQ ID NO: 3 at any other locations upstream of nucleotide 972 and the reverse primer has a sequence complementary to the nucleotides of SEQ ID NO: 3 at any other locations downstream of nucleotide 977.

23. The method of claim 16, wherein the forward primer has a sequence the nucleotides of SEQ ID NO: 5 at any other locations upstream of nucleotide 1186 and the reverse primer has a sequence complementary to the nucleotides of SEQ ID NO: 5 at any other locations downstream of nucleotide 1236.

24. The method of claim 16, wherein the forward primer has a sequence the nucleotides of SEQ ID NO: 7 at any other locations upstream of nucleotide 972 and the reverse primer has a sequence complementary to the nucleotides of SEQ ID NO: 7 at any other locations downstream of nucleotide 1176.

25. The method of claim 21, the cDNA sample amplified from SEQ ID NO: 1 is 132 bp shorter than that from SMAPK3.

26. The method of claim 22, the cDNA sample amplified from SEQ ID NO: 3 is 60 bp shorter than that from SMAPK3.

27. The method of claim 23, the cDNA sample amplified from SEQ ID NO: 5 is 51 bp longer than that from SMAPK3.

28. The method of claim 24, the cDNA sample amplified from SEQ ID NO: 7 is 9 bp shorter than that from SMAPK3.

29. The method of claim 16 further comprising the step of detecting the amount of the amplified cDNA sample.

30. (Cancel)

31. (Cancel)

32. A method for diagnosing the diseases associated with the deficiency of the SMAPK3 gene in a mammal, in particular cancers, which comprises detecting the polypeptide of claim 1.

33. The method of claim 32, wherein the detection of the polypeptide comprises the steps of contacting an antibody specifically binding to an isolated polypeptide comprising an amino acid sequence selected from the group of SEQ ID NOS: 2 and 4, and fragments thereof.

34. The method of claim 33 further comprising the step of determining the amount of the antibody-polypeptide complex.


SEQUENCE LISTING (8 sequences)

SEQ ID NO: 1 - DNA, 1654 bp, Homo sapiens
gaggagtggagatggcggcggcggcggctcaggggggcgggggcggggagccccgtagaaccgagggggtcggcccgggggtcccgggggaggtggagatggtgaaggggcagccgttcgacgtgggcccgcgctacacgcagttgcagtacatcggcgagggcgcgtacggcatggtcagctcggcctatgaccacgtgcgcaagactcgcgtggccatcaagaagatcagccccttcgaacatcagacctactgccagcgcacgctccgggagatccagatcctgctgcgcttccgccatgagaatgtcatcggcatccgagacattctgcgggcgtccaccctggaagccatgagagatgtctacattgtgcaggacctgatggagactgacctgtacaagttgctgaaaagccagcagctgagcaatgaccatatctgctacttcctctaccagatcctgcggggcctcaagtacatccactccgccaacgtgctccaccgagatctaaagccctccaacctgctcatcaacaccacctgcgaccttaagatttgtgatttcggcctggcccggattgccgatcctgagcatgaccacaccggcttcctgacggagtatgtggctacgcgctggtaccgggccccagagatcatgctgaactccaagggctataccaagtccatcgacatctggtctgtgggctgcattctggctgagatgctctctaaccggcccatcttccctggcaagcactacctggatcagctcaaccacattctggcccttgacctgctggaccggatgttaacctttaaccccaataaacggatcacagtggaggaagcgctggctcacccctacctggagcagtactatgacccgacggatgagccagtggccgaggagcccttcaccttcgccatggagctggatgacctacctaaggagcggctgaaggagctcatcttccaggagacagcacgcttccagcccggagtgctggaggccccctagcccagacagacatctctgcaccctggggcctggacctgcctcctgcctgcccctctcccgccagactgttagaaaatggacactgtgcccagcccggaccttggcagcccaggccggggtggagcatgggcctggccacctctctcctttgctgaggcctccagcttcaggcaggccaaggccttctcctccccacccgccctccccacggggcctcgggagctcaggtggccccagttcaatctcccgctgctgctgctgcgcccttaccttccccagcgtcccagtctctggcagttctggaatggaagggttctggctgccccaacctgctgaagggcagaggtggagggtggggggcgctgagtagggactcagggccatgcctgcccccctcatctcattcaaaccccaccctagtttccctgaaggaacattccttagtctcaagggctagcatccctgaggagccaggccgggccgaatcccctccctgtcaaagctgtcacttcgcgtgccctcgctgcttctgtgtgtggtgagcagaagtggagctggggggcgtggagagcccggcgcccctgccacctccctgacccgtctaatatataaatatagagatgtgtctatggctg

SEQ ID NO: 2 - protein, 335 aa, Homo sapiens
MAAAAAQGGGGGEPRRTEGVGPGVPGEVEMVKGQPFDVGPRYTQLQYIGEGAYGMVSSAYDHVRKTRVAIKKISPFEHQTYCQRTLREIQILLRFRHENVIGIRDILRASTLEAMRDVYIVQDLMETDLYKLLKSQQLSNDHICYFLYQILRGLKYIHSANVLHRDLKPSNLLINTTCDLKICDFGLARIADPEHDHTGFLTEYVATRWYRAPEIMLNSKGYTKSIDIWSVGCILAEMLSNRPIFPGKHYLDQLNHILALDLLDRMLTFNPNKRITVEEALAHPYLEQYYDPTDEPVAEEPFTFAMELDDLPKERLKELIFQETARFQPGVLEAP

SEQ ID NO: 3 - DNA, 1726 bp, Homo sapiens
gaggagtggagatggcggcggcggcggctcaggggggcgggggcggggagccccgtagaaccgagggggtcggcccgggggtcccgggggaggtggagatggtgaaggggcagccgttcgacgtgggcccgcgctacacgcagttgcagtacatcggcgagggcgcgtacggcatggtcagctcggcctatgaccacgtgcgcaagactcgcgtggccatcaagaagatcagccccttcgaacatcagacctactgccagcgcacgctccgggagatccagatcctgctgcgcttccgccatgagaatgtcatcggcatccgagacattctgcgggcgtccaccctggaagccatgagagatgtctacattgtgcaggacctgatggagactgacctgtacaagttgctgaaaagccagcagctgagcaatgaccatatctgctacttcctctaccagatcctgcggggcctcaagtacatccactccgccaacgtgctccaccgagatctaaagccctccaacctgctcatcaacaccacctgcgaccttaagatttgtgatttcggcctggcccggattgccgatcctgagcatgaccacaccggcttcctgacggagtatgtggctacgcgctggtaccgggccccagagatcatgctgaactccaagggctataccaagtccatcgacatctggtctgtgggctgcattctggctgagatgctctctaaccggcccatcttccctggcaagcactacctggatcagctcaaccacattctgggcatcctgggctccccatcccaggaggacctgaattgtatcatcaacatgaaggcccgaaactacctacagtctctgccctccaagaccaaggtggcttgggccaagcttttccccaagtcagactccaaagcccttgacctgctggaccggatgttaacctttaaccccaataaacggatcacagtggccgaggagcccttcaccttcgccatggagctggatgacctacctaaggagcggctgaaggagctcatcttccaggagacagcacgcttccagcccggagtgctggaggccccctagcccagacagacatctctgcaccctggggcctggacctgcctcctgcctgcccctctcccgccagactgttagaaaatggacactgtgcccagcccggaccttggcagcccaggccggggtggagcatgggcctggccacctctctcctttgctgaggcctccagcttcaggcaggccaaggccttctcctccccacccgccctccccacggggcctcgggagctcaggtggccccagttcaatctcccgctgctgctgctgcgcccttaccttccccagcgtcccagtctctggcagttctggaatggaagggttctggctgccccaacctgctgaagggcagaggtggagggtggggggcgctgagtagggactcagggccatgcctgcccccctcatctcattcaaaccccaccctagtttccctgaaggaacattccttagtctcaagggctagcatccctgaggagccaggccgggccgaatcccctccctgtcaaagctgtcacttcgcgtgccctcgctgcttctgtgtgtggtgagcagaagtggagctggggggcgtggagagcccggcgcccctgccacctccctgacccgtctaatatataaatatagagatgtgtctatggctg

SEQ ID NO: 4 - protein, 359 aa, Homo sapiens
MAAAAAQGGGGGEPRRTEGVGPGVPGEVEMVKGQPFDVGPRYTQLQYIGEGAYGMVSSAYDHVRKTRVAIKKISPFEHQTYCQRTLREIQILLRFRHENVIGIRDILRASTLEAMRDVYIVQDLMETDLYKLLKSQQLSNDHICYFLYQILRGLKYIHSANVLHRDLKPSNLLINTTCDLKICDFGLARIADPEHDHTGFLTEYVATRWYRAPEIMLNSKGYTKSIDIWSVGCILAEMLSNRPIFPGKHYLDQLNHILGILGSPSQEDLNCIINMKARNYLQSLPSKTKVAWAKLFPKSDSKALDLLDRMLTFNPNKRITVAEEPFTFAMELDDLPKERLKELIFQETARFQPGVLEAP

SEQ ID NO: 5 - DNA, 1837 bp, Homo sapiens
gaggagtggagatggcggcggcggcggctcaggggggcgggggcggggagccccgtagaaccgagggggtcggcccgggggtcccgggggaggtggagatggtgaaggggcagccgttcgacgtgggcccgcgctacacgcagttgcagtacatcggcgagggcgcgtacggcatggtcagctcggcctatgaccacgtgcgcaagactcgcgtggccatcaagaagatcagccccttcgaacatcagacctactgccagcgcacgctccgggagatccagatcctgctgcgcttccgccatgagaatgtcatcggcatccgagacattctgcgggcgtccaccctggaagccatgagagatgtctacattgtgcaggacctgatggagactgacctgtacaagttgctgaaaagccagcagctgagcaatgaccatatctgctacttcctctaccagatcctgcggggcctcaagtacatccactccgccaacgtgctccaccgagatctaaagccctccaacctgctcatcaacaccacctgcgaccttaagatttgtgatttcggcctggcccggattgccgatcctgagcatgaccacaccggcttcctgacggagtatgtggctacgcgctggtaccgggccccagagatcatgctgaactccaagggctataccaagtccatcgacatctggtctgtgggctgcattctggctgagatgctctctaaccggcccatcttccctggcaagcactacctggatcagctcaaccacattctgggcatcctgggctccccatcccaggaggacctgaattgtatcatcaacatgaaggcccgaaactacctacagtctctgccctccaagaccaaggtggcttgggccaagcttttccccaagtcagactccaaagcccttgacctgctggaccggatgttaacctttaaccccaataaacggatcacagtggaggaagcgctggctcacccctacctggagcagtactatgacccgacggatgagccagtggccgaggagcccttcaccttcgccatggagctggatgacctacctaaggagcggctgaaggagctcatcttccaggagacagcacgcttccagcccggagtgctggaggccccctagcccagacagacatctctgcaccctggggcctggaacagaactggcaaagaggcaagaggtcactgagggcctctgtcacccaggacctgcctcctgcctgcccctctcccgccagactgttagaaaatggacactgtgcccagcccggaccttggcagcccaggccggggtggagcatgggcctggccacctctctcctttgctgaggcctccagcttcaggcaggccaaggccttctcctccccacccgccctccccacggggcctcgggagctcaggtggccccagttcaatctcccgctgctgctgctgcgcccttaccttccccagcgtcccagtctctggcagttctggaatggaagggttctggctgccccaacctgctgaagggcagaggtggagggtggggggcgctgagtagggactcagggccatgcctgcccccctcatctcattcaaaccccaccctagtttccctgaaggaacattccttagtctcaagggctagcatccctgaggagccaggccgggccgaatcccctccctgtcaaagctgtcacttcgcgtgccctcgctgcttctgtgtgtggtgagcagaagtggagctggggggcgtggagagcccggcgcccctgccacctccctgacccgtctaatatataaatatagagatgtgtctatggctg

SEQ ID NO: 6 - protein, 379 aa, Homo sapiens
MAAAAAQGGGGGEPRRTEGVGPGVPGEVEMVKGQPFDVGPRYTQLQYIGEGAYGMVSSAYDHVRKTRVAIKKISPFEHQTYCQRTLREIQILLRFRHENVIGIRDILRASTLEAMRDVYIVQDLMETDLYKLLKSQQLSNDHICYFLYQILRGLKYIHSANVLHRDLKPSNLLINTTCDLKICDFGLARIADPEHDHTGFLTEYVATRWYRAPEIMLNSKGYTKSIDIWSVGCILAEMLSNRPIFPGKHYLDQLNHILGILGSPSQEDLNCIINMKARNYLQSLPSKTKVAWAKLFPKSDSKALDLLDRMLTFNPNKRITVEEALAHPYLEQYYDPTDEPVAEEPFTFAMELDDLPKERLKELIFQETARFQPGVLEAP

SEQ ID NO: 7 - DNA, 1777 bp, Homo sapiens
gaggagtggagatggcggcggcggcggctcaggggggcgggggcggggagccccgtagaaccgagggggtcggcccgggggtcccgggggaggtggagatggtgaaggggcagccgttcgacgtgggcccgcgctacacgcagttgcagtacatcggcgagggcgcgtacggcatggtcagctcggcctatgaccacgtgcgcaagactcgcgtggccatcaagaagatcagccccttcgaacatcagacctactgccagcgcacgctccgggagatccagatcctgctgcgcttccgccatgagaatgtcatcggcatccgagacattctgcgggcgtccaccctggaagccatgagagatgtctacattgtgcaggacctgatggagactgacctgtacaagttgctgaaaagccagcagctgagcaatgaccatatctgctacttcctctaccagatcctgcggggcctcaagtacatccactccgccaacgtgctccaccgagatctaaagccctccaacctgctcatcaacaccacctgcgaccttaagatttgtgatttcggcctggcccggattgccgatcctgagcatgaccacaccggcttcctgacggagtatgtggctacgcgctggtaccgggccccagagatcatgctgaactccaagggctataccaagtccatcgacatctggtctgtgggctgcattctggctgagatgctctctaaccggcccatcttccctggcaagcactacctggatcagctcaaccacattctgggcatcctgggctccccatcccaggaggacctgaattgtatcatcaacatgaaggcccgaaactacctacagtctctgccctccaagaccaaggtggcttgggccaagcttttccccaagtcagactccaaagcccttgacctgctggaccggatgttaacctttaaccccaataaacggatcacagtggccgaggagcccttcaccttcgccatggagctggatgacctacctaaggagcggctgaaggagctcatcttccaggagacagcacgcttccagcccggagtgctggaggccccctagcccagacagacatctctgcaccctggggcctggaacagaactggcaaagaggcaagaggtcactgagggcctctgtcacccaggacctgcctcctgcctgcccctctcccgccagactgttagaaaatggacactgtgcccagcccggaccttggcagcccaggccggggtggagcatgggcctggccacctctctcctttgctgaggcctccagcttcaggcaggccaaggccttctcctccccacccgccctccccacggggcctcgggagctcaggtggccccagttcaatctcccgctgctgctgctgcgcccttaccttccccagcgtcccagtctctggcagttctggaatggaagggttctggctgccccaacctgctgaagggcagaggtggagggtggggggcgctgagtagggactcagggccatgcctgcccccctcatctcattcaaaccccaccctagtttccctgaaggaacattccttagtctcaagggctagcatccctgaggagccaggccgggccgaatcccctccctgtcaaagctgtcacttcgcgtgccctcgctgcttctgtgtgtggtgagcagaagtggagctggggggcgtggagagcccggcgcccctgccacctccctgacccgtctaatatataaatatagagatgtgtctatggctg

SEQ ID NO: 8 - protein, 359 aa, Homo sapiens; identical to SEQ ID NO: 4